(19) United States
(12) Patent Application Publication
(10) Pub. No.: US 2022/0117920 A1
(43) Pub. Date: Apr. 21, 2022

(54) N-Aryl Benzenesulfonamides as Protonophores for the Treatment of Cancers, Metabolic Diseases and Traumatic Brain Injury

(71) Applicants: University of Kentucky Research Foundation, Lexington, KY (US); Yale University, New Haven, CT (US)

(72) Inventors: David S Watt, Lexington, KY (US); Roberto Gedaly, Lexington, KY (US); Brett T. Spear, Lexington, KY (US); Yang Yang-Hartwich, Hamden, CT (US); Chunming Liu, Lexington, KY (US); Francesc Marti, Lexington, KY (US); Patrick Sullivan, Nicholasville, KY (US); Wen Zhang, Lexington, KY (US)

(21) Appl. No.: 17/431,408

(22) PCT Filed: Feb. 14, 2020
(86) PCT No.: PCT/US20/18431

§ 371 Date: Aug. 16, 2021

Related U.S. Application Data
(60) Provisional application No. 62/805,800, filed on Feb. 14, 2019.

(51) Int. Cl. A61K 31/18; A61P 35/00

(52) U.S. Cl. CPC A61K 31/18; A61P 35/00

(57) ABSTRACT

Provided herein are methods for treating a disease, such as cancer, the methods including administering one or more N-aryl benezenesulfonamides or analogs thereof to a subject in need thereof.

### SEQUENCE LISTING

The instant application contains a Sequence Listing which has been submitted in ASCII format via EFS-Web and is hereby incorporated by reference in its entirety. The ASCII copy of the Sequence Listing, which was created on Feb. 14, 2020, is named 13177N-2349US.txt and is 3 kilobytes in size.

### TECHNICAL FIELD

The presently-disclosed subject matter generally relates to compositions and methods for treatment of cancer, bacterial disease, metabolic disease, and/or traumatic brain injury. In particular, certain embodiments of the presently-disclosed subject matter relate to N-aryl benzenesulfonamides and methods for treating diseases using the same.

### BACKGROUND

The electron-transport chain (ETC) drives proton translocation to the intermembrane space in the mitochondria and this process, in turn, drives ATP synthesis. Disruption of this process by compounds capable of inhibiting complexes I-IV in the ETC, or by compounds capable of disrupting the proton/electrochemical gradient across the inner mitochondrial membrane, has a long and storied history.

Compounds that disrupt the latter process are described as “proton uncouplers” or “protonophores” and transport protons in the opposite direction to the pumping mechanisms associated with complexes I-IV. The compound most frequently associated with the latter process is 2,4-dinitrophenol (DNP) (1) (FIG. 1). Munitions workers in factories manufacturing explosives during WWI suffered exposure to DNP and lost weight. This observation led ultimately to the marketing of DNP as a weight-reduction drug, but subsequent deaths led to the creation of the Food and Drug Administration and the withdrawal of DNP from the market. The rapid dissipation of the proton/electrochemical gradient led to the generation of heat, elevated body temperature and deaths. The negative outcomes with DNP, until recently, led to a generalized association of “proton uncouplers” with unwanted toxicity.

Recently, however, several authors suggested that small-molecule proton uncouplers have unappreciated therapeutic potential if investigators find an appropriate “window” between therapeutic utility and toxicity. A number of papers point to potential applications for the treatment of bacterial diseases, cancer, obesity, and other metabolic conditions. Unfortunately, though, existing proton uncouplers suffer from safety and efficacy concerns.

Accordingly, there remains a need for safe and effective proton uncouplers to be used in therapeutic applications.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

In some embodiments, the presently-disclosed subject matter includes a method of treating a disease, the method comprising administering one or more N-aryl benezenesulfonamides or analogs thereof to a subject in need thereof. In some embodiments, the subject is a human subject. In some embodiments, the N-aryl benzenesulfonamide is a proton uncoupler. In some embodiments, the N-aryl benzenesulfonamide is a halogenated N-aryl benzenesulfonamide. In some embodiments, the N-aryl benzenesulfonamide is selected from the group consisting of 2,5-dichloro-N-(2-methyl-4-nitrophenyl)benzenesulfonamide (FH535), 2,5-dichloro-N-(4-nitronaphthalen-1-yl)benzenesulfonamide (Y3), analogs thereof, and combinations thereof. In some embodiments, the N-aryl benzenesulfonamide is Y3.

In some embodiments, the disease is cancer. In one embodiment, the cancer is characterized by aberrant Wnt/β-catenin signaling. In another embodiment, the cancer is selected from the group consisting of hepatocellular cancer, colorectal cancer, or a combination thereof. In some embodiments, the cancer is ovarian cancer. In some embodiments, the disease is traumatic brain injury. In some embodiments, the disease is a bacterial disease. In some embodiments, the disease is a metabolic disease.

In some embodiments, the one or more N-aryl benezenesulfonamides or analogs thereof are administered as part of a composition. In some embodiments, the composition comprises the one or more N-aryl benezenesulfonamides or analogs thereof and a pharmaceutically acceptable solvent or carrier.

Further features and advantages of the presently-disclosed subject matter will become evident to those of ordinary skill in the art after a study of the description, figures, and non-limiting examples in this document.

### BRIEF DESCRIPTION OF THE DRAWINGS

FIG. 1 shows the structure of 2,4-Dinitrophenol, an early example of a protonophore.

FIG. 2 shows representative examples of N,N′-diarylureas (2) and N-aryl benzenesulfonamides (3).

FIG. 3 shows structures of N-aryl benzenesulfonamides Y3 (4) and Y3-M (5).

FIG. 4 shows a schematic illustrating proton transport across the inner mitochondrial membrane by “Y3.”

FIGS. 5A-C show images and graphs illustrating identification of urea derivatives as novel Wnt inhibitors. (A) Structures of 1-(1,1,1,4,4,4-hexafluoro-2-(trifluoromethyl)butan-2-yl)-3-(5-(trifluoromethyl)-1,3,4-thiadiazol-2-yl)urea (FTU-11) and 1-(4-(trifluoromethyl)phenyl)-3-(3,4,5-trifluorophenyl)urea (FDN-4E). (B-C) Dose-response luciferase study using FTU-11 and FDN-4E in a HEK293T cell line containing TOPFlash reporter. (D) Effects of FTU-11 and FDN-4E in HEK293T cells transfected with Super 8× TOFFlash or 8× FOPFlash.

FIGS. 6A-B show graphs and images validating antineoplastic activity and Wnt inhibition activity of FTU-11 and FDN-4E. (A) Cell proliferation assays using FTU-11 and FDN-4E in LS174T and DLD-1 CRC cells. (B) Dose response study of FTU-11 and FDN-4E on components of Wnt signaling pathway and downstream targets.

FIG. 7 shows images illustrating that FTU-11 and FDN-4E are activated AMPK in CRC cells. FTU-11 (3 μM) and FDN-4E (3 μM) increased AMPK activity (T172 phosphorylation) and inhibited ACC (S79 phosphorylation).

FIGS. 8A-E show graphs and images illustrating a linkage between AMPK activation and the inhibition of Wnt signaling. (A) Compound C (5 μM), an AMPK inhibitor, decreased urea-induced AMPK phosphorylation and ACC phosphorylation. (B) Failure of Compound C (5 μM) to rescue Wnt signaling inhibited by FTU-11 (3 μM) and FDN-4E (3 μM). Failure of a specific AMPK activator, A769662 (10 μM), to inhibit Wnt signaling. (C) Increased AMPK activity caused by A769662 (ACC S79 phosphorylation) without affecting AMPK phosphorylation. (D) FH535 (3 μM) inhibited Wnt signaling. (E) FH535 (3 μM) activated AMPK.

FIGS. 9A-F show graphs and images illustrating the effects of FTU-11 and FH535 on mitochondrial respiration. (A) FTU-11 (3 μM) and FH535 (3 μM) and reduced mitochondrial ATP production using mitochondrial uncoupler FCCP (1 μM) as a control. Glycolytic ATP production rates were increased upon uncoupler treatment. (B) FTU-11 (3 μM) reduced mitochondrial membrane potential. (C) Uncoupling assay: model of uncoupling effects on Oxygen Consumption rate (OCR). (D-F) Effects of FCCP (1 μM) or testing compound (3 μM) in uncoupling assays in DLD-1 cells.

FIGS. 10A-F show a graphs and images illustrating that uncoupler function is linked to AMPK activation and Wnt inhibition. (A) 2,5-dichloro-N-(2-methyl-4-nitrophenyl)benzenesulfonamide (FH535), it active analog Y3 and their N-methylation analog FH535-M and Y3-M. (B) FH535-M (3 μM) failure to induce uncoupling activity (red: FH535; Grey: FH535-M; and Blue: DMSO). (C-F) FH535-M (3 μM) and Y3-M (3 μM) lost activities in inducing AMPK activation and inhibiting Wnt signaling.

FIGS. 11A-C show graphs and images illustrating that FH535 and Y3 directly target mitochondria. (A) Schematic representation of proton uncoupling promoted by FH535. (B-C) Uncoupling activities of FCCP (1 μM) and test compounds in purified mitochondria from mouse liver.

FIGS. 12A-F show graphs and images illustrating a common mechanism of mitochondria uncoupler and glycolytic inhibitor on AMPK activation and Wnt signaling inhibition. (A-B) Mitochondrial uncoupler FCCP activated AMPK and inhibited Wnt signaling. (C-D) Glycolytic inhibitor 2-DG activated AMPK, inhibited Wnt signaling. (E) Effects of 2-DG (10 mM) on ATP levels in LS174T cells. (F) FTU-11 and FH535 reduced LiCl-induced β-catenin accumulation in LS174T cells.

FIG. 13 shows a graph illustrating a HPLC trace for compound FTU-11

FIG. 14 shows a graph illustrating a HPLC trace for compound FDN-4E

FIG. 15 shows a graph illustrating a HPLC trace for compound FH535

FIG. 16 shows a graph illustrating a HPLC trace for compound FH535-M

FIG. 17 shows a graph illustrating a HPLC trace for compound Y3

FIG. 18 shows a graph illustrating a HPLC trace for compound Y3-M

FIG. 19 shows images illustrating the structures of FH535 (2,5-dichloro-N-(2-methyl-4-nitrophenyl)benzenesulfonamide) and FH535-N(2,5-dichloro-N-(4-nitronaphthalen-1-yl)benzenesulfonamide).

FIGS. 20A-D show graphs and images illustrating FH535 effect in vivo. (A) Mice body weight after FH535 treatment. C57BL/6 mice (n=5) were treated by intraperitoneal injection with 15 mg/Kg of FH535 or DMSO vehicle control every 4 days for 6 weeks. Mice were monitored before injections for signs of body weight loss, impaired mobility, labored breathing and body score based on the Ullman-Culleré MH, Foltz CJ method. (B-D) FH535 reduces tumor growth in vivo in a xenograft tumor model. Huh7 cell were injected subcutaneously on the right flank of athymic nude mice. FH535 (15 mg/Kg) or vehicle (DMSO) were administrated by intraperitoneal injection every other day when tumor size reached 100 mm³. (B) Tumor growth was monitored every other day until day 10 of starting treatments when mice were euthanized according to the AVMA guidelines, *p<0.05 (n=5, each group); (C) Tumor weight of excised tumors after 10 day treatment with FH535 reduced the tumor weight in 42±8% compared to vehicle treatment, **p<0.001 (n=4, each group). (D) H&E and ki67 stainings from one representative tumor of each group treatment. Pictures were taken at 400× magnification. H&E stainings showed poorly differentiated carcinoma comprised of sheets of epithelioid cells with increased N/C ratio, enlarged nuclei with prominent nucleoli, high mitotic activity and tumor necrosis (lower right corner of the picture for FH535, and left upper corner and left mid area of the picture for control group). The Ki-67 immunohistochemical staining highlights very high mitotic index with nuclear staining in more than 95% of the viable neoplastic cells for both groups.

FIGS. 21A-C show graphs and images illustrating that FH535 regulates autophagic activity in HCC cells. Western blot analysis of Huh7 cell after 40 h treatment with FH535 at indicated concentrations in absence (−CQ) or presence (+CQ) of 50 μM chloroquine for 8 h. (A) LC3B and (B) p62 were used as autophagy markers for western blot analysis. Band intensity were estimated using Image? software. Autophagic flux was determined by subtracting the band intensity of LC3B II western blot in presence of CQ and the corresponding treatment in absence of CQ which is referred as ΔLC3II (LC3II (+CQ)−LC3II (−CQ)) (A, right panel). (C) mRNA p62 expression levels were assessed by RT-qPCR in absence of CQ.

FIG. 22 shows graphs and images illustrating that β-catenin knockdown induced changes in LC3II and p62 protein levels in Huh7 cells. Western blot analysis of LC3BII and p62 protein levels of Huh7 transiently transfected with a β-catenin (β-cat) or control (Ctrl) siRNA.

FIG. 23 shows graphs illustrating that FH535 regulates autophagic flux in Huh7 cells. Autophagic activity of Huh7 cells after 40 h FH535 treatment in absence (−CQ) or presence (+CQ) of 50 μM CQ (8 h) was determined by flow cytometry analysis using the Cyto-ID autophagy detection reagent. Results are shown as GeoMean±SD from viables cells (Zombie negative population). Autophagic flux was determined by the difference in Geomean between cells treated with CQ and corresponding treatment in absence of CQ also referred as ΔGeoMean (GeoMean (+CQ)−GeoMean (−CQ)) (right panel). *: p<0.05.

FIG. 24 shows graphs illustrating the effect of FH535-N on HCC cells proliferation. Cell proliferation was measured on Huh7, PLC/PRF/5 and Hep3B cells using 3H-thymidine incorporation after 72 h treatment with FH535 or FH535-N alone or in combination with sorafenib at the concentrations indicated. Results are represented as mean±SD, n=4. *: p<0.05, **p<0.001.

FIGS. 25A-D show the effect FH535-N on inhibition of Wnt/β-catenin pathway. (A) Effect of FH535-N on TOPFlash activation. Huh7 cells were co-transfected with Top-Flash and phRL-TK plasmid. After 5 h of transfection, cells were treated with vehicle, FH535 or FH535-N at the concentration indicated in presence of 10 mM LiCl. Vehicle in absence of LiCl was used as control for basal levels of Wnt/β-catenin activity. Results are represented as mean±SD, n=3. #: p<0.05, *: p<0.001. (B) Effect of FH535-N on expression of β-catenin targets. Protein expression levels of downstream β-catenin targets from Huh7 cells treated with FH535-N for 36 h were determined by western blot analysis (left panel). Densitrometry analysis was performed using ImageJ software (right panel). (C) mRNA expression of downstream β-catenin targets from Huh7 cells treated with FH535-N for 36 h were determined by RT-qPCR.

FIGS. 26A-B show graphs illustrating the effect of FH535-N alone or in combination with sorafenib on apoptosis of HCC cells. Analysis of apoptosis by Annexin V-APC/propidium iodide (PI) double staining of HuH7 and PLC/PRF/5 cells after 48 h treatment at the concentration of FH535, FH535-N and sorafenib indicated. (A) Two-color flow cytometry dot plots show the percentages of living cells as negative for both annexin V and PI; early-stage apoptotic cells as the populations testing Annexin V positive and PI negative, and late-stage apoptotic/necrotic cells as double-positive cells. (B) Results are represented as mean±SD, n=3. *: p<0.05, **: p<0.001.

FIG. 27 shows graphs illustrating that mitochondrial respiration changes induced after 24 h-treatment of Huh7 cells with FH535, FH535-N alone or in combination with sorafenib. Representative OCR profiles of Huh7 cells shown as percentage change with respect to the OCR levels after addition of the ATP-synthase inhibitor Oligomycin (0). The parameters of ATP turnover, proton leak, and spare respiratory capacity were calculated as area under the curve (AUC) values as described in Materials and Methods section. Data are shown as mean±SEM, n=6-8.*: p<0.05 and **: p<0.001. Statistical comparisons were performed using one-way ANOVA and Dunnett's multiple comparisons test and pairwise comparisons with Student's t test. (NS=non-significant; p>0.05).

FIGS. 28A-B show graphs and images illustrating that FH535-N regulates autophagic activity in HCC cells. Western blot analysis of Huh7 cell after 40 h treatment with FH535-N at indicated concentrations in absence (−CQ) or presence (+CQ) of 50 μM chloroquine for 8 h. (A) Protein expression levels of LC3BII. Autophagic flux was determined by subtracting the band intensity of LC3B II western blot in presence of CQ and the corresponding treatment in absence of CQ which is referred as ΔT C3II (LC3II (+CQ)−LC3II (−CQ)). (B) Protein expression levels of p62 in absence of CQ Band intensity from Western blots were estimated using ImageJ software.

FIG. 29 shows graphs illustrating that FH535-N regulates autophagic flux in Huh7 cells. Autophagic activity of Huh7 cells after 40 h FH535 treatment in absence (−CQ) or presence (+CQ) of 50 μM CQ (8 h) was determined by flow cytometry analysis using the Cyto-ID autophagy detection reagent. Results are shown as GeoMean±SD from viables cells (Zombie negative population). Autophagic flux was determined by the difference in Geomean between cells treated with CQ and corresponding treatment in absence of CQ also referred as ΔGeoMean (GeoMean (+CQ)−GeoMean (−CQ) (right panel). *: p<0.05.

FIG. 30 shows graphs illustrating that FH535-N regulates autophagic flux in Huh7 and PLC/PRF/5 cells. Autophagic activity of Huh7 cells after 40 h FH535 treatment in absence (−CQ) or presence (+CQ) of 50 μM CQ (8 h) was determined by flow cytometry analysis using the Cyto-ID autophagy detection reagent. Results are shown as GeoMean±SD from viables cells (Zombie negative population). Autophagic flux was determined by the difference in GeoMean between cells treated with CQ and corresponding treatment in absence of CQ also referred as ΔGeoMean (GeoMean (+CQ)−GeoMean (−CQ) (right panel).*: p<0.05 and **: p<0.001. (NS=non-significant; p>0.05).

FIGS. 31A-B show images and a graph illustrating ICD inducer for cancer immunotherapy. (A) Mechanisms of novel ICD inducer SF-Y3 and its analogs. (B) Vaccination assay. C57BL/6 mice vaccinated with SF-Y3 treated TKO cells did not develop tumors as the controls did.

FIGS. 32A-E show graphs illustrating that SF-Y3 induces apoptosis of EOC cells. (A) Cell viability after 48h treatment assessed by Celltiter Glo Assay. (B) Flow cytometry histograms of A2780 cells stained by mitochondrial membrane potential dyes, JC-1 and TMRE. (C) Flow cytometry detected p-S6 protein levels. MFI, median fluorescence intensity. (D) Apoptotic cells detected by AnnexinV/PI staining and flow cytometry. (E) Caspase 3/7 activity in A2780 cells after 48h treatment was assessed using Caspase-3/7 Glo Assay. *p<0.05, ***p<0.0005.

FIGS. 33A-D show images and graphs illustrating tumor inhibiting effect of SF-Y3 in nude mice. (A) Representative images of xenograft tumors. Red fluorescence protein (RFP)-labeled 5×10\hat{ }6 patient-derived EOC cells were IP injected to nude mice. Three days later, nude mice were injected with vehicle or SF-Y3 (20 mg/kg) twice per week and imaged with IVIS system. (B) Tumor RFP signal was quantified using IVIS system and analysis software. (C) Total tumor weight and tumor numbers of IP tumors from each mouse were calculated. (D) Caspase-9 and 3 activity in 10 μg tumor protein lysate was assessed using Caspase-9 or 3 Glo Assay. *p<0.05, **p<0.005, ***p<0.0005, #p<0.0001.

FIGS. 34A-G show graphs and images illustrating bioactivity of SFs. (A) Chemical structures. (B) Model schematic. (C) Mitochondrial and glycolytic ATP production rate in DLD-1 cells was analyzed using Agilent Seahorse XF Real-Time ATP Rate Assay. (D) Oxygen consumption rate (OCR) was measured using Seahorse Analyzer in colon cancer (DLD-1) and ovarian cancer (OVC-8) cell lines. (E) LS174T cell lysate was analyzed by Western blot. (F) TOPFlash reporter assay. (G) OCR of purified mitochondria from mouse liver.

FIG. 35 shows a schematic illustrating immunogenic cell death (ICD). DCs, dendritic cells. CTL, cytotoxic T lymphocytes. CALR, calreticulin.

FIGS. 36A-C show graphs illustrating that SF-Y3 induces DAMPs emission. (A) ATP in the medium of A2780 cells was assessed using luminescence-based ATP assay. (B) HMGB1 in the medium of A2780 cells was assessed by ELISA. (C) Cell surface CALR was detected by flow cytometry.

FIGS. 37A-G show graphs and images illustrating that SF-Y3 induces ER stress response. (A) RT-QPCR of ER stress response genes. (B) ER stress response pathway. (C) Co-immunoprecipitation of Bip1 and three ER stress sensors in A2780 cells. (D) Western blot of ATF6. (E) Western blot of p-elF1α. (F) RT-PCR and gel analysis of XBP1 mRNA splicing. (G) RNA expression of tumors from nude mice IP injected with vehicle or SF-Y3. *p<0.05, **p<0.005, ***p<0.0005, #p<0.0001, unpaired student's t-test.

FIG. 38 shows an image and a graph illustrating SF-Y3 treatment in syngeneic EOC mouse model. TKO cells (10\hat{ }7) were SC injected to C57BL/6 mice. When tumors reached ˜30 mm³, they were treated with the vehicle (PEG:DMSO:saline=7:2:1) or 5 mg/kg SF-Y3 SC injection once a week. Tumor size=3.14/6*length*width*width. (n=5, P<0.001).

FIG. 39 shows graphs illustrating cytokine secretion of C57BL/6 mice. SF-Y3 (5 mg/kg IP twice per week) or vehicle was injected to healthy mice or TKO tumor-bearing mice. The levels of 31 cytokines in the blood plasma were analyzed using a multiplex assay. (Healthy mice, n=10/group. Cancer mice, n=5/group. *p<0.05, **p<0.005).

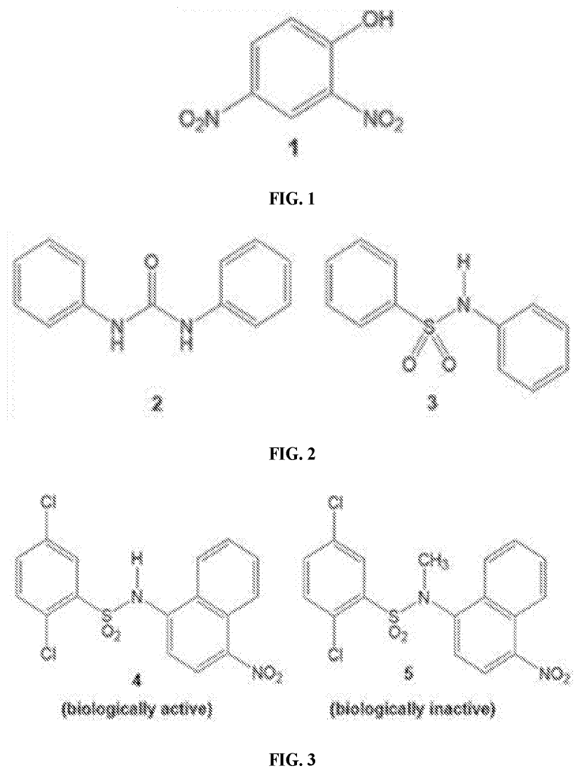

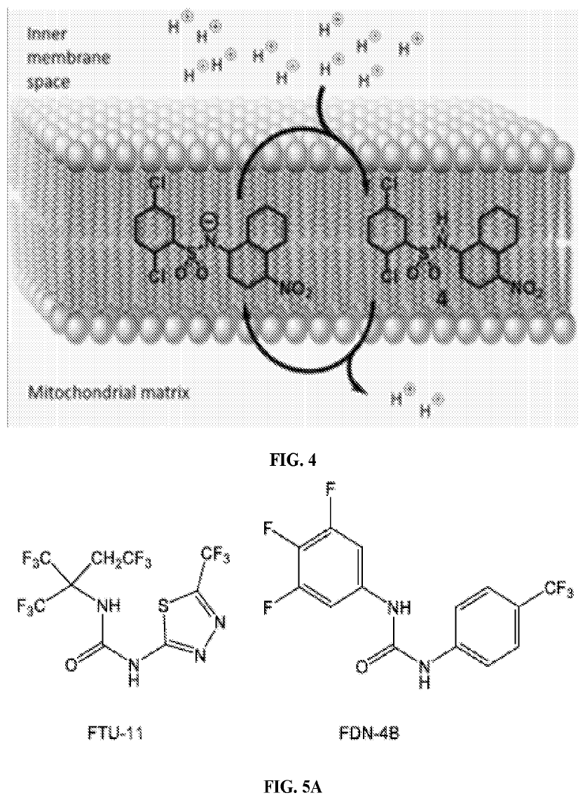

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Unless defined otherwise, all technical and scientific terms used herein have the same meaning as is commonly understood by one of skill in the art to which the invention(s) belong. All patents, patent applications, published applications and publications, GenBank sequences, databases, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more disorders, e.g., uncontrolled cellular proliferation or a traumatic brain injury prior to the administering step. In some aspects of the disclosed method, the subject has been diagnosed with a disorder of uncontrolled cellular proliferation, e.g., a cancer, prior to the administering step. In some aspects of the disclosed method, the subject has been identified with a disorder treatable by proton uncoupling prior to the administering step. In some aspects of the disclosed method, the subject has been identified with a disorder treatable by small-molecule immunogenic cell-death (ICD) inducers prior to the administering step. In some aspects of the disclosed method, the subject has been identified with a bacterial or viral infection prior to the administering step. In some aspects of the disclosed method, the subject has been identified with a traumatic brain injury. In one aspect, a subject can be treated prophylactically with a compound or composition disclosed herein, as discussed herein elsewhere.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

The term “pharmaceutically acceptable” describes a material that is not biologically or otherwise undesirable, i.e., without causing an unacceptable level of undesirable biological effects or interacting in a deleterious manner.

As used herein, the terms “derivative” and “analog” refer to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

Provided herein are compounds for the treatment of one or more diseases. In some embodiments, the compound includes a protonophore. In some embodiments, the compound includes an N-aryl benzenesulfonamide (3; FIG. 2) or analog thereof. For example, in some embodiments, the N-aryl benzenesulfonamide includes a halogenated N-aryl benzenesulfonamide such as, but not limited to, 2,5-dichloro-N-(2-methyl-4-nitrophenyl)benzenesulfonamide (FH535), 2,5-dichloro-N-(4-nitronaphthalen-1-yl)benzenesulfonamide, which is also referred to herein as “Y3” (4; FIG. 3), analogs thereof, or a combination thereof. In some embodiments, unlike existing N,N′-diarylureas, one or more of the compounds disclosed herein exhibited minimal toxicity while maintaining good activity. For example, the halogenated N-aryl benzenesulfonamides, such as Y3, were potent protonophores. In view of the existing belief that proton uncouplers exhibited unwanted toxicity that rendered them unsuitable for use as therapeutics, this potent protonophore activity of the N-aryl benzenesulfonamides with minimal toxicity is both surprising and unexpected.

Also provided herein, in some embodiments, are methods of treating a disease. In some embodiments, the method includes administering one or more of the compounds disclosed herein to a patient in need thereof. Without wishing to be bound by theory, as discussed in detail in the Examples below, it is believed that the compounds disclosed herein, such as the N-aryl benzenesulfonamides, disrupt ATP synthesis, trigger the activation of the energy sensor AMP-dependent protein kinase (AMPK), and independently starve ATP-dependent signaling pathways. These biologically active N-aryl benezenesulfonamides affect multiple signaling pathways that are disregulated in carcinomas: RAS/RAF/MAPK, PI3K/Akt/mTOR, HGF/c-MET, IGF, VEGF, PDGF and Wnt/β-catenin. The latter signaling pathway represents an existing challenge in the case of hepatocellular and colorectal carcinomas where aberrant Wnt/β-catenin appears in approximately one-third of cases. Accordingly, in some embodiments, the methods include administering on or more of the compounds disclosed herein to treat hepatocellular and/or colorectal carcinomas. Additionally or alternatively, the compounds disclosed herein may increase endoplasmic reticulum stress. Accordingly, in some embodiments, the methods include administering on or more of the compounds disclosed herein to treat ovarian cancer.

The present inventors have also identified an immunomodulation connection between proton uncoupling and disruption of Treg function that can be exploited in cancer treatment by modulating the tumor microenvironment. Furthermore, the present inventors have discovered that N-aryl benezenesulfonamides may be used to treat traumatic brain injury. For example, the classic protonophore, 2,4-dinitrophenol, exhibited some promising activity in animal models. Accordingly, diseases which may be treated by the compounds and methods disclosed herein include, but are not limited to, cancers, bacterial diseases, metabolic diseases, traumatic brain injury, or a combination thereof.

Still further, the present inventors have discovered that one or more of the compounds disclosed herein form small-molecule immunogenic cell-death (ICD) inducers. For example, in some embodiments, Y3 forms an ICD inducer, providing an additional route to treat diseases such as cancer with the compounds disclosed herein.

Further provided herein, in some embodiments, is a composition for treating a disease. In some embodiments, the composition includes one or more of the compounds disclosed herein and a pharmaceutically acceptable solvent and/or carrier.

The presently-disclosed subject matter is further illustrated by the following specific but non-limiting examples. The following examples may include compilations of data that are representative of data gathered at various times during the course of development and experimentation related to the presently-disclosed subject matter.

### EXAMPLES

### Example 1

An Underlying Mechanism of Dual Wnt Inhibition and AMPK Activation: Mitochondrial Uncouplers Masquerading as Wnt Inhibitors

Abstract

The importance of upregulated Wnt signaling in colorectal cancers led to efforts to develop inhibitors that target β-catenin in this pathway. We now report that several “Wnt inhibitors” that allegedly target β-catenin actually function as mitochondrial proton uncouplers that independently activate AMPK and concomitantly inhibit Wnt signaling. As expected for a process in which mitochondrial uncoupling diminishes ATP production, a mitochondrial proton uncoupler, FCCP, and a glucose metabolic inhibitor, 2-DG, activated AMPK and inhibited Wnt signaling. Also consistent with these findings, a well-known “Wnt inhibitor”, FH535, functioned as a proton uncoupler, and in support of this finding, the N-methylated analog, 2,5-dichloro-N-methyl-N-(2-methyl-4-nitrophenyl)benzenesulfonamide (FH535-M) was inactive as an uncoupler and Wnt inhibitor. Apart from suggesting an opportunity to develop dual Wnt inhibitors and AMPK activators, these findings provide a cautionary tale that claims for Wnt inhibition alone require scrutiny as possible mitochondrial proton uncouplers or inhibitors of the electron transport chain.

Introduction

Aberrant activation of Wnt signaling is a hallmark of many human cancers, particularly colorectal cancer (CRC), and the development of inhibitors that target this pathway and the re-purposing of non-cancer related, FDA-approved drugs that target this pathway represent promising venues for therapeutic advances in cancer treatment. The Wnt inhibitors in current use include “off-label” drugs and new agents now under evaluation in Phase 1/2 clinical trials. The mechanisms by which these agents affect the Wnt signaling pathway are often unclear, and efforts to understand these events at a biochemical level will facilitate the development of future Wnt inhibitors.

In the absence of ligands that trigger Wnt signaling, β-catenin undergoes sequential phosphorylation by casein kinase-1 alpha (CK1a) and glycogen synthase kinase-3 (GSK3) in the Axin/Adenomatous Polyposis Coli complex (Axin/APC) to furnish phosphorylated β-catenin. In normal cells, phosphorylated β-catenin undergoes ubiquitination by β-Transducin Repeat Containing E3 Ubiquitin Protein Ligase (β-TrCP) and subsequent proteasomal degradation. In colorectal cancer cells, mutations in either β-catenin or APC render this phosphorylation-ubiquitination-degradation sequence dysfunctional, and either β-catenin phosphorylation or 13-TrCP-mediated degradation fail. These outcomes lead to aberrant β-catenin accumulation and the translocation of undesired levels of β-catenin to the nucleus. In the nucleus, these augmented levels of β-catenin bind to co-activators, including the T-cell factor/lymphoid enhancer factor (TCF/LEF), induce transcription of Wnt-target genes, such as c-Myc and cyclin D1, and promote undesired growth.

The complexity of the Wnt pathway lends itself to the development of inhibitors that target various stages of these signaling events. High-throughput-screening methods using stable cell lines containing Wnt reporter genes find frequent application for siRNA or in small molecule screening. Porcupine inhibitors, IWP-2 or LGK974, block the secretion of Wnt ligands that initiate the signaling cascade and promote β-catenin degradation. Tankyrase inhibitors, XAV939 or IWR-1, stabilize Axin and also promote β-catenin degradation. Other inhibitors, such as ICG-001, bind and inhibit CREB-binding Protein (CBP), a transcriptional co-activator of the β-catenin/TCF complex, and inhibit Wnt target gene expression. In contrast, several, alleged Wnt inhibitors lack direct targets or clear mechanisms but find widespread use by many investigators in research focused on the Wnt pathway or cancer biology.

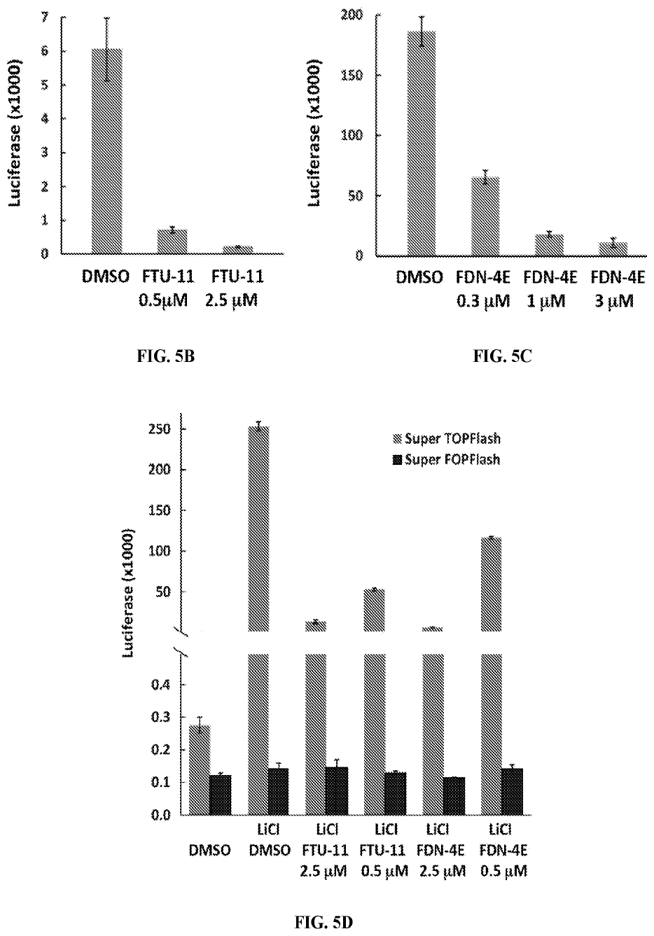

We studied alleged Wnt inhibitors in several chemical classes (e.g., N,N-diarylureas, N-arylbenzenesulfonamides) but failed, despite considerable effort, to elucidate precise biological targets associated directly with the Wnt signaling pathway using biologically active biotinylated analogs and pull-down assays. For example, after screening a library of more than 5,000 compounds using a stable HEK293T cell line containing a modified TOPFlash reporter we identified 1-(1,1,1,4,4,4-hexafluoro-2-(trifluoromethyl)butan-2-yl)-3-(5-(trifluoromethyl)-1,3,4-thiadiazol-2-yl)urea (FTU-11) (FIG. 5A) as a potential Wnt inhibitor. We noted that FTU-11 resembled another fluorinated urea, 1-(4-(trifluoromethyl)phenyl)-3-(3,4,5-trifluorophenyl)urea (FDN-4E) (FIG. 5A), reported to function as an AMPK activator. Our chance observations that FDN-4E also inhibited Wnt signaling and that FTU-11 also activated AMPK signaling suggested a common mechanism for urea-mediated, Wnt inhibition and AMPK activation. As an additional example, the N-aryl benezenesulfonamide, 2,5-dichloro-N-(2-methyl-4-nitrophenyl)benzenesulfonamide (FH535), reported as an inhibitor of both β-catenin in the Wnt signaling pathway and the Peroxisome Proliferator-activated Receptor (PPAR), also activated AMPK. A confluence of screening, and analog development along several lines led a fortuitous intersection of experiments in which we recognized a linkage among the Wnt pathway, AMPK activation, and oxidative phosphorylation. We identified several soi-disant Wnt inhibitors that actually function as proton uncouplers or electron-transport inhibitors.

Results

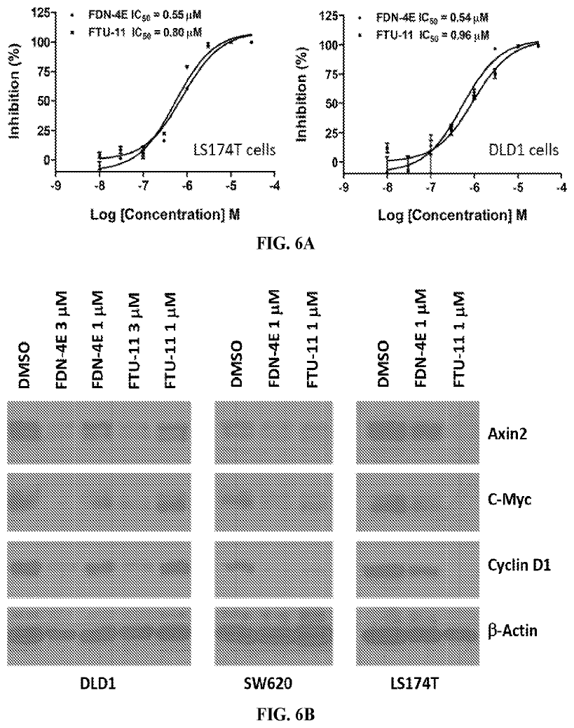

To identify novel Wnt regulators by high throughput screening, a stable HEK293T cell line containing the TOPFlash reporter was established. To identify Wnt inhibitors that targeted molecular events downstream of β-catenin, we treated these cells with lithium chloride to inhibit GSK3 and to stabilize β-catenin. Screening a library previously available from the Drug Discovery Center at University of Cincinnati (Cincinnati, Ohio, USA) led to the identification of FTU-11 (FIG. 5A) that inhibited Wnt signaling at a 0.5 mM concentration (96-well assay using stable cell line) (FIG. 5B). FTU-11 possessed structural features that resembled another highly fluorinated urea, FDN-4E (FIG. 5A), that functioned as a potent AMPK activator and repressed the growth of CRC cells. A comparison study of FTU-11 and FDN-4E using the luciferase assay revealed that FDN-4E also inhibited Wnt signaling (24-well assay using stable cell line) (FIG. 5C). We also validated the results by transient transfection of Super 8× TOPFlash or 8× FOPFlash into HEK293T cells. Both FTU-11 and FDN-4E inhibited TOPFlash but not FOPFlash activity (FIG. 5D). Since the results from the stable and transient transfection are compatible, we used stable cell lines for all of the other experiments. In addition, the reporter activity, FTU-11 and FDN-4E also inhibited the proliferation of colon cancer cells at sub-micromolar concentrations (FIG. 6A) and inhibited Wnt target genes in three CRC cell lines (FIG. 6B).

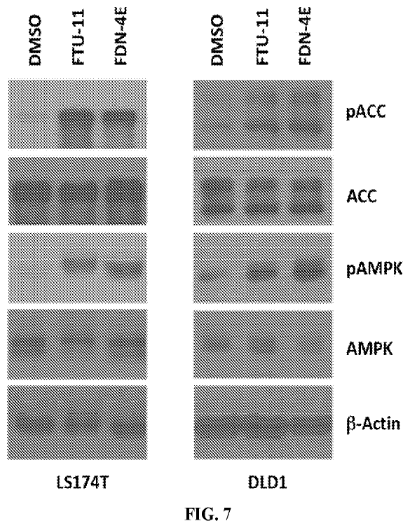

Most importantly, we observed that FTU-11 functioned as an AMPK activator equal in potency to FDN-4E, and as expected for an AMPK activator, FTU-11 inhibited acetyl-CoA carboxylase (ACC) that was subject to regulation by phosphorylated AMPK (FIG. 7). Taken together, these findings suggested a linkage between AMPK activation and the inhibition of Wnt signaling. We probed this relationship using a series of AMPK inhibitors and activators as well as inhibitors of the Wnt pathway in which we accepted at face value the assertions in the literature that these Wnt inhibitors and AMPK activators had exclusive selectivity for one of these targets. As a working hypothesis, we assumed a direct relationship in which FTU-11 and FDN-4E disrupted some cellular process that triggered AMPK activation. The phosphorylated AMPK in turn served as an inhibitor of the ATP-dependent Wnt pathway.

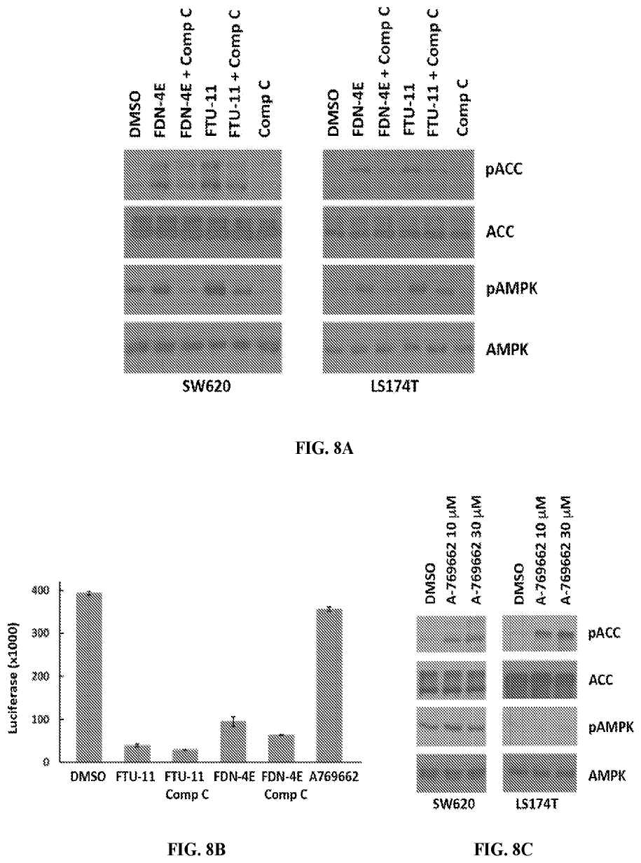

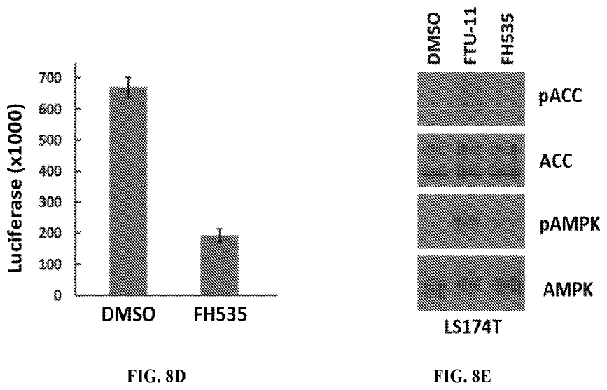

We treated HEK293T cells containing a modified TOPFlash reporter simultaneously with either FTU-11 or FDN-4E and with a potent AMPK inhibitor, 4-(2-(4-(3-(pyridin-4-yl)pyrazolo[1,5-a]pyrimidin-6-yl)phenoxy)ethyl)morpholine, known commonly as “Compound C.” If the inhibition of Wnt signaling required activated, phosphorylated AMPK, as proposed in our hypothesis, then Compound C should rescue the Wnt signaling inhibited by either FTU-11 or FDN-4E. Compound C inhibited ACC phosphorylation (i.e., conversion to its inactive form), as expected (FIG. 8A), but it had no effect on either the FTU-11-mediated or FDN-4E-mediated Wnt inhibition (FIG. 8B). Next, we treated HEK293T cells containing a modified TOPFlash reporter with an AMPK activator, 4-hydroxy-3-(2′-hydroxy-[1,1′-biphenyl]-4-yl)-6-oxo-6,7-dihydrothieno[2,3-b]pyridine-5-carbonitrile (A769662) (FIG. 8C), but unlike the results using FTU-11 and FDN-4E, the direct AMPK activator, A769662, did not inhibit Wnt signaling (FIG. 8B). Taken together, these results suggested that the ureas, FTU-11 and FDN-4E, inhibited Wnt signaling through an AMPK-independent mechanism.

We hypothesized that either FTU-11 or FDN-4E affected a cellular process that altered Wnt signaling and independently triggered the activation of the AMPK energy sensor. We tested well-known Wnt inhibitors, such as (6S,9aS)—N-benzyl-6-(4-hydroxybenzyl)-8-(naphthalen-1-ylmethyl)-4,7-dioxohexahydro-2H-pyrazino[1,2-c]pyrimidine-1(6H)-carboxamide (ICG-001) and FH535. These compounds had dramatically different effects on AMPK activation. ICG-001 only inhibited Wnt signaling but had no effect on AMPK (data not shown), and in contrast, FH535 not only inhibited Wnt signaling (FIG. 8D) but also induced AMPK phosphorylation and ACC phosphorylation (FIG. 8E). Since ICG-001 was a well-characterized Wnt inhibitor with a specific target, namely the CREB Binding Protein (CBP), we concluded that FTU-11 and FH535 activated AMPK through Wnt-independent mechanisms. In addition, we detected AMPK activation within 15 min of treatment with FH535. This rapid response was inconsistent with AMPK regulation mediated by Wnt transcription. In conclusion, AMPK activation was an unlikely a consequence of Wnt inhibition.

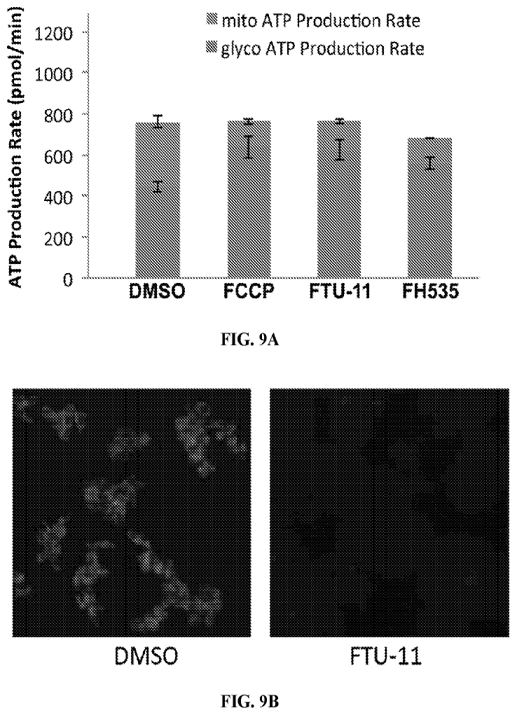

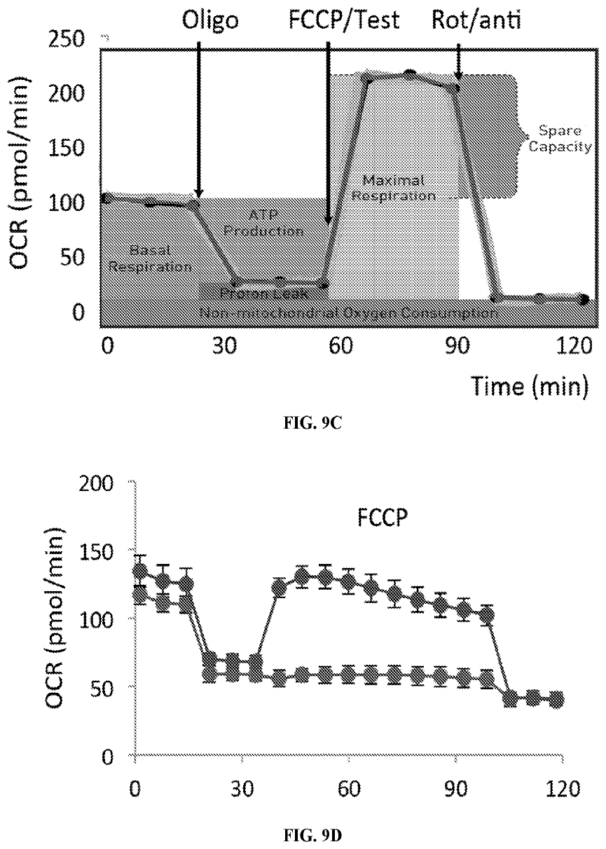

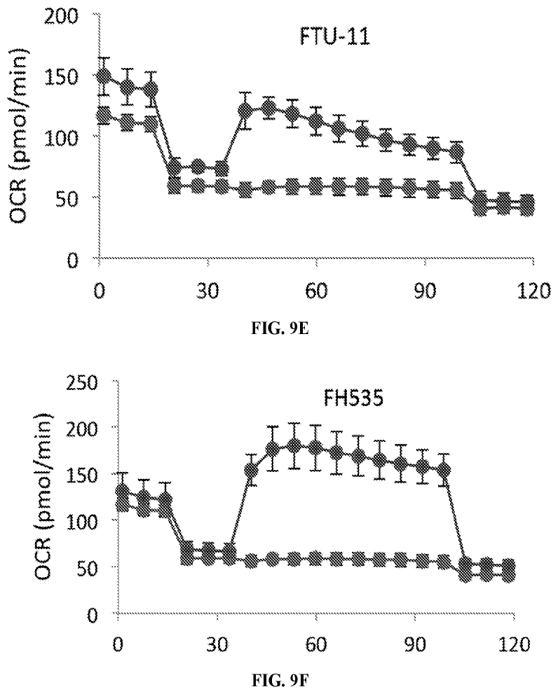

Since either FTU-11 or FH535, but not A769662, inhibited Wnt signaling, we concluded that these N,N-diarylureas indirectly activated AMPK through a mechanism that altered AMP/ATP ratio. To confirm this point, we analyzed ATP production in FTU-11-treated and FH535-treated cells using a Seashorse XF Analyzer. As a control, we selected a well-known mitochondrial uncoupler, N-(4-(trifluoromethoxy)phenyl)carbonohydrazonoyl dicyanide (FCCP), and we found that FTU-11, FH535, and FCCP decreased the rates of ATP production in mitochondria (FIG. 9A). As expected, the decrease in mitochondrial ATP production led to concomitant increase in glycolytic ATP production and AMPK activation. These results suggested that these compounds induced AMPK activation by inhibiting mitochondrial function.

FCCP represented a potent, mitochondrial uncoupler that translocated protons across the mitochondrial inner membrane, decreased oxidative phosphorylation that provided ATP and increased oxygen consumption rate (OCR) as a means of compensating for the increased pH in the intermembrane space. FCCP possessed both a hydrophobic substructure and an ionizable nitrogen-hydrogen bond with a pKₐ sufficient to function as a transmembrane proton transporter. Since uncouplers reduced membrane potential, we analyzed the effect of FTU-11 on mitochondrial membrane potential using tetramethylrhodamine methyl ester (TMRM) staining and found that FTC-11 significantly reduced the membrane potential of mitochondria (FIG. 9B). FDN-4E and FH535 had similar effects (data not shown).

A previous study indicated that FH535 affected mitochondria respiration, but the exact mechanism was unclear. The above data suggested that the ureas in this study and other, soi-disant Wnt inhibitors functioned as mitochondrial proton uncouplers. We next analyzed the effects of these compounds on OCR using Seashorse XF Analyzer, with FCCP as a control (FIGS. 9C-D). Oligomycin (Oligo) inhibited ATPase and thus inhibited OCR. FCCP, FTC-11, and FH535 uncoupled mitochondrial oxidation/phosphorylation (OXPHOS) and increased OCR inhibited by oligomycin (FIGS. 9E-F). Rotenone and antimycin A blocked the increase in OCR by inhibiting electron-transport Complexes I and III, respectively.

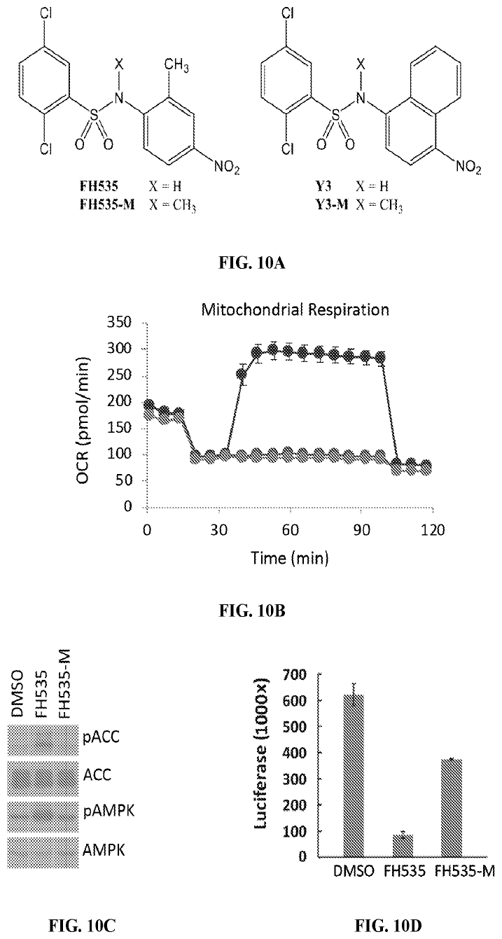

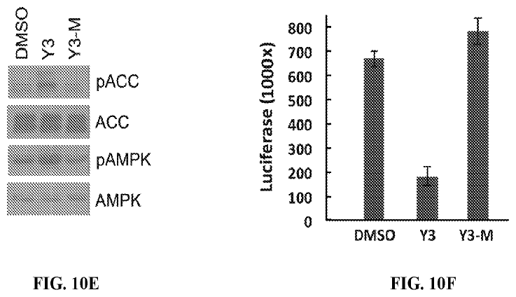

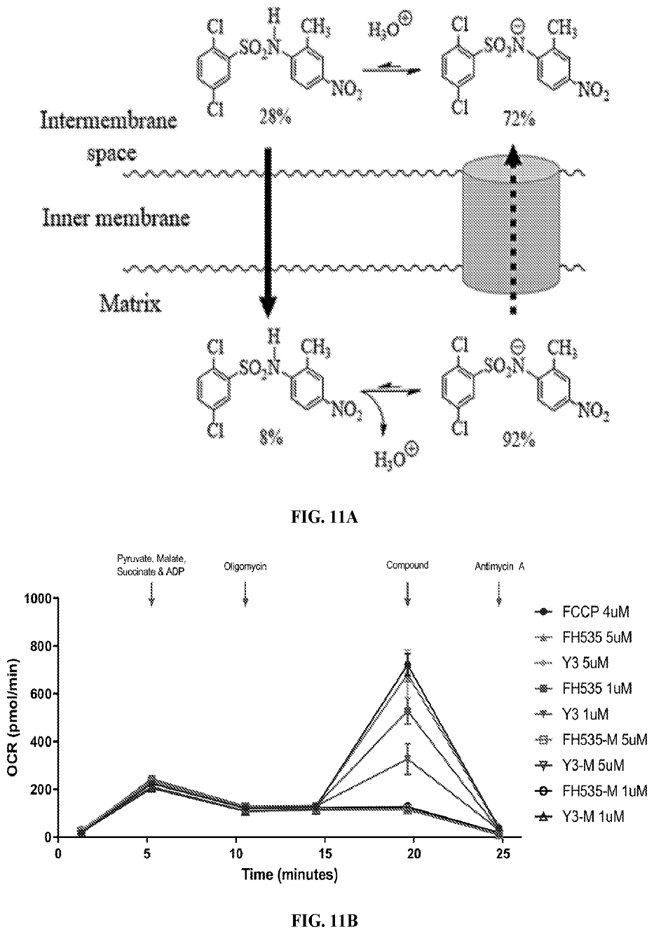

In the uncoupling assay, FH535 had the same function as the standard mitochondrial uncoupler, FCCP. As was the case for FCCP, FH535 also possessed a hydrophobic substructure and an ionizable, nitrogen-hydrogen bond that participated in proton translocation. To confirm this point, we replaced this “active” hydrogen with a methyl group (FIG. 10A), and as expected, we found that the N-methyl analog, 2,5-dichloro-N-methyl-N-(2-methyl-4-nitrophenyl)benzenesulfonamide (FH535-M), no longer functioned as a mitochondrial uncoupler (FIG. 10B) and did not activate AMPK (FIG. 10C) or inhibit Wnt (FIG. 10D). Furthermore, we synthesized FH535 analogs and identified 2,5-dichloro-N-(4-nitronaphthalen-1-yl)benzenesulfonamide analog, which we called Y3 (FIG. 10A), that strongly activated AMPK and inhibited Wnt signaling (FIGS. 10E-F). Just as in the case of FH535 and FH535-M, the methylated version of Y3, 2,5-dichloro-N-methyl-N-(4-nitronaphthalen-1-yl)benzenesulfonamide (Y3-M) (FIG. 10A), failed as a mitochondrial uncoupler (data not shown) and led to neither AMPK activation (FIG. 10E) or Wnt inhibition (FIG. 10F).

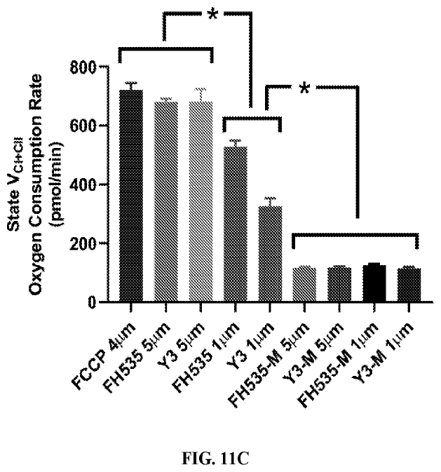

The calculated pKₐ values for FH535 and Y3 using the ACD/pKₐ DB (version 11.1) software (Advanced Chemistry Development, Inc., Toronto, Ontario, Canada) were 5.83 and 5.39, respectively, and the calculated pKₐ values for FH535 and Y3 using the ChemAxon (version 19.18) software (ChemAxon, Inc., Cambridge, Mass.) were 6.69 and 6.46, respectively. In support of the validity of these pKₐ values, the calculated values for another sulfonamide, trifluoromethanesulfonamide (CF₃SO₂NH₂), using these same two programs were 6.37 and 6.19, respectively, and these values compared well with the experimentally determined value of 6.3. Based on the reported pH values for the intermembrane space (pH 6.88±0.09) and the mitochondrial matrix (pH 7.78±0.17) and assuming the release of these uncouplers into these two compartments and not simply the retention in the inner membrane, the ChemAxon pKₐ values predicted that the percentages of the protonated forms of FH535 in the hydronium ion-rich intermembrane space and the mitochondrial matrix were 39% and 8%, respectively (FIG. 11A). In the same fashion, the percentages of the protonated forms of Y3 in the hydronium ion-rich intermembrane space and the mitochondrial matrix were 28% and 5%, respectively.

A model for proton translocation entails the transport of the uncoupler and the conjugate base of the uncoupler across the inner mitochondrial membrane. As suggested diagrammatically (FIG. 11A), the inner membrane possesses several transporters for the translocation of other anionic species (e.g., Pᵢ, ADP), and these channels may participate in translocating the conjugate base. In summary, these calculated pKₐ values and pH values for the intermembrane space and matrix of mitochondria were consistent with both FH535 and Y3 retaining reasonable, equilibrium concentrations of protonated and unprotonated forms sufficient to translocate protons across the inner membrane. In addition, FH535 and Y3 possessed calculated log P values (i.e., 5.83 and 5.39, respectively), and these values were similar to other mitochondrial proton uncouplers, again consistent with representing FH535 and Y3 as weak acids possessing the necessary lipophilicity to accomplish uncoupling. These results further supported that the mitochondrial proton uncoupling activity of these compounds contributed to their activities as AMPK activators and Wnt inhibitors.

Since the above studies were performed in human cell lines, it was not clear whether the test compounds targeted mitochondria directly, or indirectly through cell signaling pathways, including the Wnt signaling pathway. To address this question, we performed uncoupling assay using purified mitochondria from mouse livers. Similar to the results in cell lines, the OCR was blocked by oligomycin and rescued by control proton uncoupler, FCCP. FH535 and Y3 also increased OCR in dose-dependent manner, but as expected, the methylated analogs, FH535-M and Y3-M were inactive as mitochondrial proton uncouplers (FIGS. 11B-C). Although we cannot rule out the possibility that these compounds may also have a mitochondrial-independent function, the experiments with purified mitochondria suggested that these “Wnt inhibitors” directly target mitochondria as proton uncouplers.

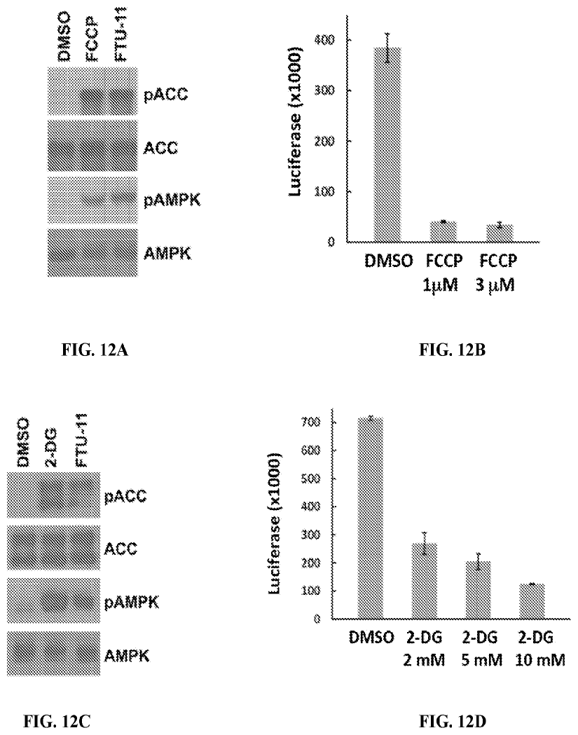

To test the hypothesis that the uncoupling effects inhibited Wnt signaling, we also analyzed a classic mitochondrial proton uncoupler, FCCP, and found that FCCP strongly activated AMPK (FIG. 12A) and inhibited Wnt signaling (FIG. 12B). We then tested numerous inhibitors in the Seahorse assay, including oligomycin, rotenone and antimycin and found that all of them inhibited oxidative phosphorylation, activated AMPK and inhibited Wnt signaling (data not shown), an observation again consistent with the inhibition of Wnt signaling by reduced ATP production. To test further this hypothesis, we treated cells with a metabolically inert, glucose analog, 2-deoxy-D-glucose (2-DG), to reduce ATP levels by inhibiting glucose metabolism, and as expected 2-DG activated AMPK (FIG. 12C), inhibited Wnt signaling (FIG. 12D) and reduced ATP levels in treated cells (FIG. 12E). We analyzed the β-catenin levels in LS174T cells. LiCl treatment increased both total and nucleus β-catenin. FTU-11 and FH535 treatment reduced β-catenin induced by LiCl (FIG. 12F), suggesting that uncouplers may affect β-catenin protein synthesis or nuclear localization.

Conclusions

Many steps in the Wnt signaling pathway, including β-catenin nuclear import and chromatin remodeling, are ATP-dependent. We found that FTU-11 and FH535 reduced β-catenin levels in LS174T cells (FIG. 12F), consisted with ATP requirements for β-catenin protein synthesis or nuclear localization. Other reports described ATP-dependent steps in Wnt signaling. For example, the ATP-dependent chromatin remodeling protein, Brg1, bound to β-catenin and activated the transcription of Wnt-target genes. Deletion of the ATPase domain of Brg1 inhibited Wnt signaling. Loss of Brg1 attenuated Wnt signaling and prevented Wnt-dependent tumorigenesis in the intestines. A recent paper suggested that impaired mitochondrial ATP production inhibited Wnt signaling via an induction of ER stress. Wnt signaling also regulates mitochondria dynamics and membrane potential in a subset of cancers. Although ATP is not specifically required for Wnt signaling, this study illuminated the linkage between this ATP requirement and claims that some compounds, previously identified as direct Wnt inhibitors, in fact function as either inhibitors of mitochondrial oxidative phosphorylation or the electron transport chain. This study not only suggested a need to re-examine some of these prior claims but also suggested a new strategy for inhibiting Wnt signaling in the development of new antineoplastic agents for cancer treatment. Many agents that function as AMPK activators may also act as Wnt inhibitors and provide an impetus to examine connections in which drugs for treating metabolic disorders find a potential application in cancer therapeutics.

Materials and Methods

(1) Chemistry

General Methods. FTU-11 was purchased from a chemical library once available through the University of Cincinnati (Cincinnati, Ohio, USA) and purified by HPLC. FDN-4E, FH535, and Y3 were synthesized as previously described. Solvents were used from commercial vendors without further purification unless otherwise noted. Nuclear magnetic resonance spectra were determined in DMSO-d6 using Varian instruments (¹H, 400; ¹³C, 100Mz; Varian, Inc., Palo Alto, Calif., USA). High resolution electrospray ionization (ESI) mass spectra were recorded on a LTQ-Orbitrap Velos mass spectrometer (Thermo Fisher Scientific, Waltham, Mass., USA). The FT resolution was set at 100,000 (at 400 m/z). Samples were introduced through direct infusion using a syringe pump with a flow rate of 5 μL/min. Melting points were determined in open capillarity tubes with a Büchi B-535 melting point apparatus (Büchi Corp., New Castle, Del., USA) and are uncorrected. Purity was established by combustion analyses performed by Atlantic Microlabs, Inc. (Norcross, Ga., USA). Further confirmation of purity of all tested compounds was obtained by RP-HPLC that was performed on an Agilent Technologies 1260 Infinity HPLC system by using the following general method: flow rate=0.5 mL/min; λ=254 nm; column=Vydac 201SP C18, 250 mm×4.6 mm, 90 Å, 5 μm. Eluent A: H₂O+0.1% TFA (v/v); Eluent B: acetonitrile; gradient profile, starting from 5% B, increasing from 5% B to 100% B over 10 min, holding at 100% B from 10 to 20 min, and decreasing from 100% B to 5% B from 20 to 23 min. Prior to each injection, the HPLC column was equilibrated for 10 min with 1% B. All compounds tested were determined to be ≥97% pure. Other chemicals were purchased from Sigma-Aldrich (St. Louis, Mo., USA) or Fisher Scientific (Pittsburgh, Pa., USA) with purify >95%.

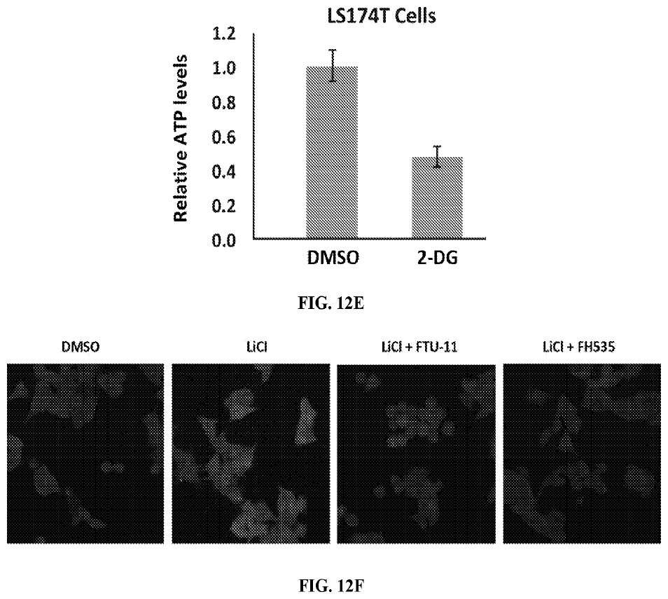

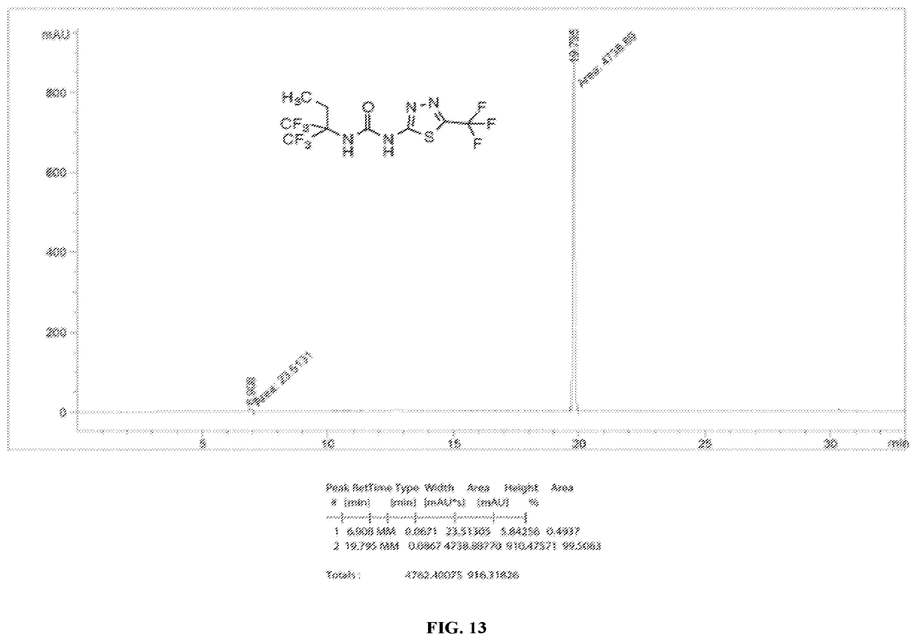

1-(1,1,1-Trifluoro-2-(trifluoromethyl)butan-2-yl)-3-(5-(trifluoromethyl)-1,3,4-thiadiazol-2-yl)urea (FTU-11). The purity of FTU-11 was confirmed by RP-HPLC: Rₜ=19.79 min (99% pure; FIG. 13).

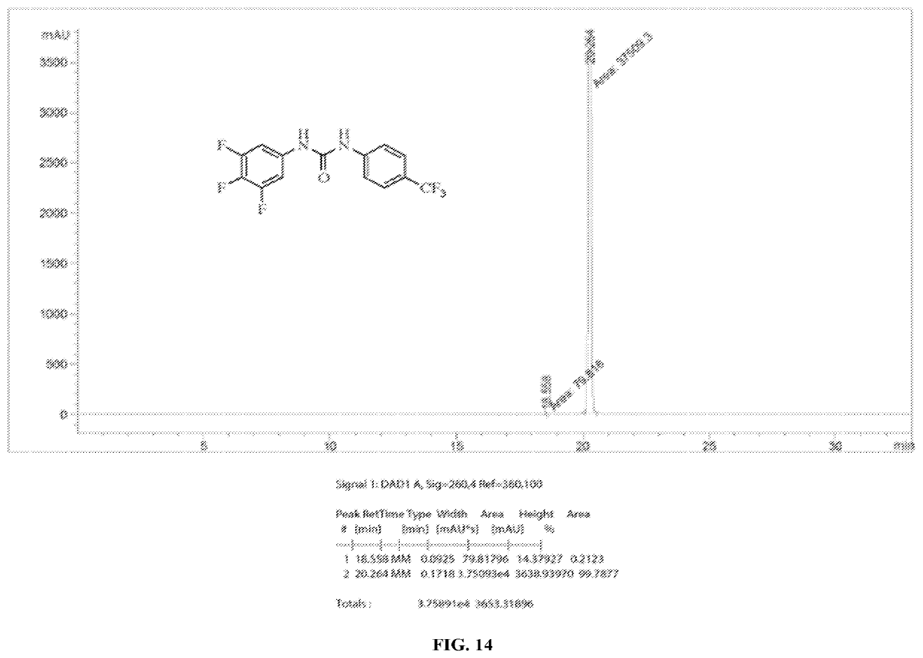

1-(4-(Trifluoromethyl)phenyl)-3-(3,4,5-trifluorophenyl)urea (FDN-4E). To a stirred solution of 77 mg (0.52 mmol) of 3,4,5-trifluoroaniline in 3 mL of benzene was added 97 mg (0.52 mmol, 1 eq) of 1-isocyanato-4-(trifluoromethyl)benzene. The mixture was stirred at 50° C. for 3 h and was cooled to 25° C. A precipitate was collected by filtration to provide 90 mg (52%) of analytically pure FDN-4E as a white solid: mp 241-242° C. ¹H NMR: δ 9.29 (s, 1H, NH), 9.14 (s, 1H, NH), 7.67 (d, 2H, J=8.8 Hz), 7.62 (d, 2H, J=8.8 Hz), 7.42-7.38 (m, 2H). ¹³C NMR: δ 152.12, 150.17 (ddd, J=243.5, 9.9, 5.6 Hz), 142.95 (d, J=1.2 Hz), 135.86 (td, J=12.1, 3.5 Hz), 135.17 (t, J=15.7 Hz), 132.75 (t, J=15.8 Hz), 128.52, 126.07 (q, J=3.8 Hz), 125.82, 123.12, 122.22 (q, J=32.0 Hz), 120.42, 118.22, 102.71 (dd). HRMS (ESI) Calcd for C₁₄H₉F₆N₂O [MH⁺]: 335.0614. Found: 335.0619. Anal. Calcd for C₁₄H₈F₆N₂O: C, 50.31; H, 2.41. Found: C, 50.09; H, 2.49. The purity of FDN-4E was further confirmed by RP-HPLC: Rₜ=20.3 min (99% pure; FIG. 14).

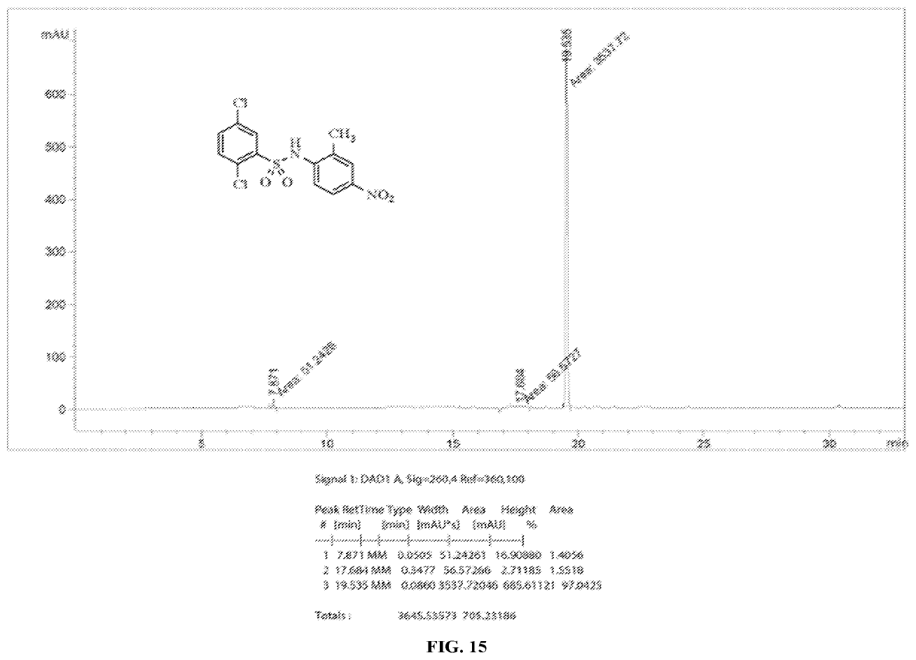

2,5-Dichloro-N-(2-methyl-4-nitrophenyl)benzenesulfonamide (FH535). This compound was synthesized as previously described. The purity of FH535 was confirmed by RP-HPLC: Rₜ=19.5 min (97% pure; FIG. 15).

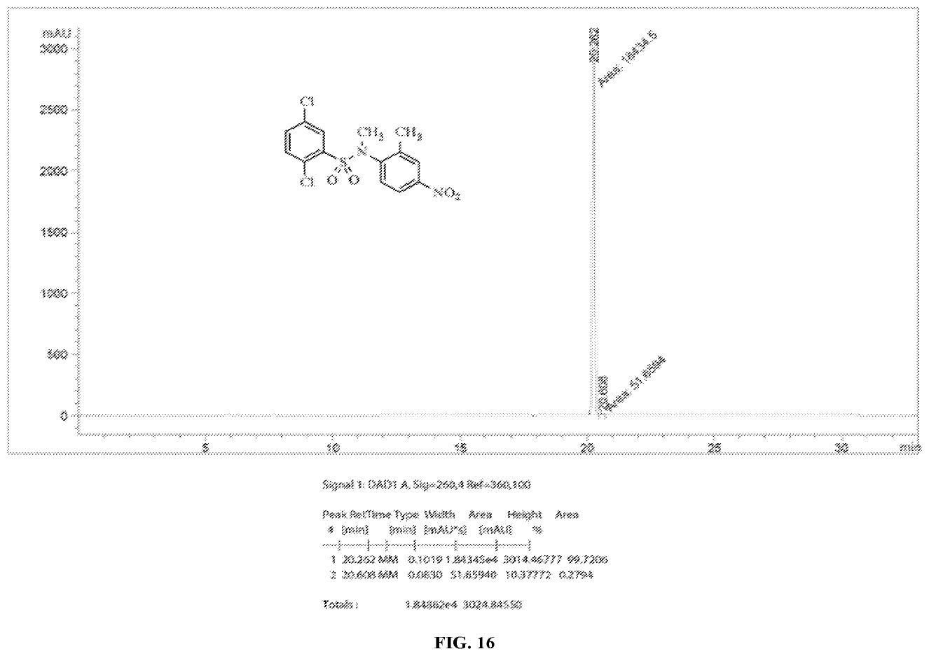

2,5-Dichloro-N-methyl-N-(2-methyl-4-nitrophenyl)benzenesulfonamide (FH535-M). To a stirred solution of 360 mg (1 mmol, 1 eq) of FH535 and 830 mg (6 mmol, 6 eq) of potassium carbonate in 2 mL of DMF was slowly added 0.12 mL (2 mmol, 2 eq) of methyl iodide. After stirring at 25° C. for 20 h, the mixture was poured into brine, extracted with dichloromethane, dried over anhydrous MgSO₄, and evaporated under reduced pressure to afford a crude product. Purification by preparative layer silica gel chromatography using 1:10 (v/v) ethyl acetate-hexane provided 0.2 g (54%) of FH-535-M: mp 142-143° C. ¹H NMR: δ 8.23 (d, J=2.7 Hz, 1H), 8.02 (dd, J=8.7, 2.8 Hz, 1H), 7.87-7.77 (m, 3H), 7.26 (d, J=8.7 Hz, 1H), 3.3 (s, 3H), 2.38 (s, 3H). ¹³C NMR: δ 146.87, 144.7, 140.33, 137.41, 134.79, 134.33, 132.38, 131.05, 130.2, 129.9, 126.05, 121.97, 39.39, 17.84. HRMS (ESI) Calcd for Ci₄H₁₃Cl₂N₂O₄S [MH+]: 374.9968. Found: 374.9968. Anal. Calcd for C₁₄H₁₂Cl₂N₂O₄S: C, 44.81; H, 3.22; N, 7.47. Found: C, 44.71; H, 3.17; N, 7.41. The purity of FI1535-M was confirmed by RP-HPLC: Rₜ=20.26 min (99% pure; FIG. 16).

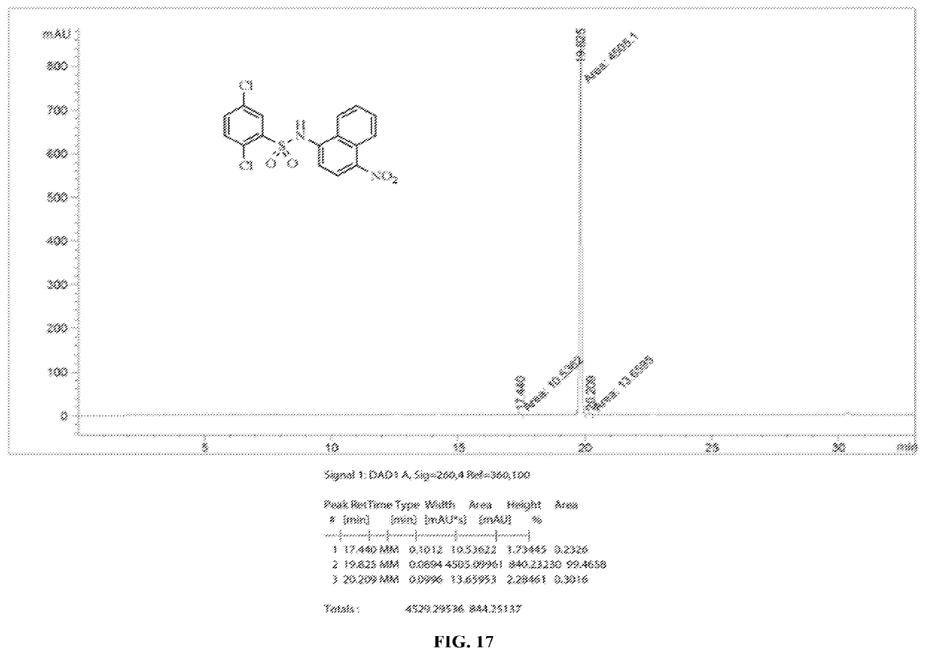

2,5-Dichloro-N-(4-nitronaphthalen-1-yl)benzenesulfonamide (Y3). To a suspension of 2.5 mmol (2.5 eq) of sodium hydride (60% dispersion in oil) in 5 mL of anhydrous tetrahydrofuran (THF) was added a solution of 188 mg (1 mmol) of 4-nitro-1-naphthylamine in 1 mL of THF. The mixture was stirred for 10 min at 0° C. and 246 mg (1 mmol) of 2,5-dichlorobenzenesulfonyl chloride was added. The mixture was stirred for 12 h at 25° C., quenched with saturated NaHCO₃ and extracted with ethyl acetate. The combined organic layers were washed with brine, dried over anhydrous MgSO₄ and concentrated in vacuum to provide crude product. Purification by recrystallization from methanol provided 254 mg (64%) of Y3: mp 232-233° C. ¹H NMR: δ 11.45 (br s, 1H), 8.41 (d, 2H, J=8.8 Hz), 8.27 (d, 1H, J=8.4 Hz), 7.94 (d, 1H, J=2.4 Hz), 7.82-7.78 (m, 1H), 7.75-7.68 (m, 3H), 7.45 (dd, 1H, J=8.4 and 2.6 Hz). ¹³C NMR: δ 143.38, 138.47, 138.11, 134.62, 133.88, 132.30, 130.35, 129.92, 129.67, 128.37, 127.62, 125.23, 124.73, 123.69, 122.74, 118.96. HRMS (ESI) Calcd for C₁₆H₉Cl₂N₂O₄S [MH−]: 394.9666. Found: 394.9662. Anal. Calcd for C₁₆H₁₀Cl₂N₂O₄S: C, 48.38; H, 2.54. Found: C, 48.63; H, 2.46. The purity of Y3 was confirmed by RP-HPLC: Rₜ=19.82 min (99% pure; FIG. 17).

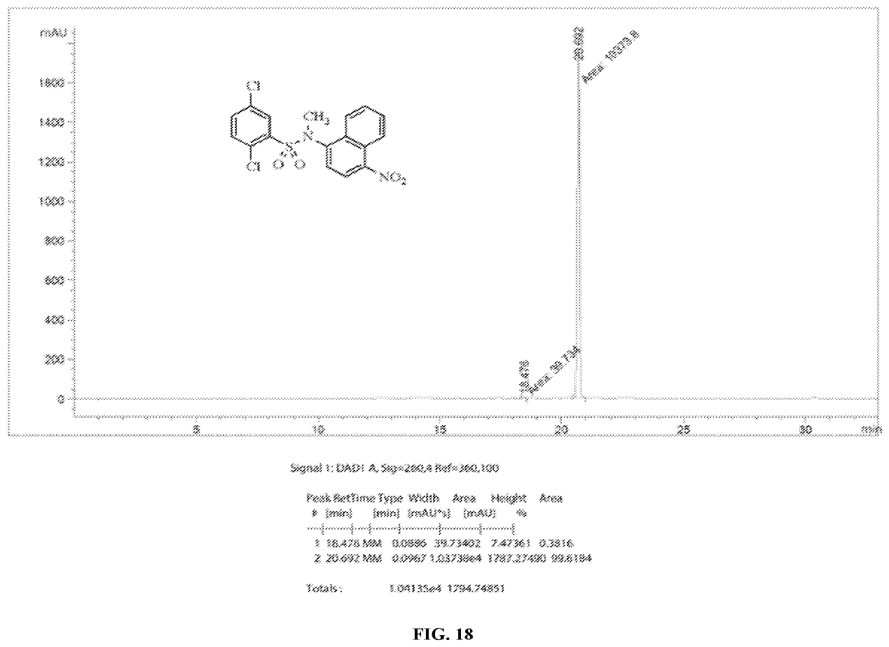

2,5-Dichloro-N-methyl-N-(4-nitronaphthalen-1-yl)benzenesulfonamide (Y3-M). To a solution of 100 mg (0.25 mmol, 1 eq.) of Y3 and 210 mg (6 mmol, 6 eq) of potassium carbonate in 2 mL of DMF was slowly added 0.031 mL (2 mmol, 2 eq) of methyl iodide. After stirring at 25° C. for 20 h, the mixture was poured into brine and extracted with dichloromethane. The combined organic layers were dried over anhydrous MgSO₄ and evaporated under reduced pressure to afford a crude product. Purification by preparative layer silica gel chromatography using 1:10 ethyl acetate-hexane (v/v) provided 70 mg (68%) of Y3-M: δ 8.32 (dd, J=8.8 Hz, 2.8 Hz, 1H), 8.25 (d, J=8 Hz, 1H), 8.21 (d, J=8 Hz, 1H), 7.86-7.8 (m, 5H), 7.46 (d, J=8 Hz, 1H), 3.48 (s, 3H). Anal. Calcd for C₁₇H₁₂Cl₂N₂O₄S: C, 49.65; H, 2.94; N, 6.81. Found: C, 49.78; H, 2.88; N, 6.73. The purity of Y3-M was confirmed by RP-HPLC: Rₜ=20.7 min (99% pure; FIG. 18).

(2) Biology

Cell culture. LS174T colon cancer cells were cultured in EMEM (ATCC, 30-2003) containing 10% (v/v) Fetal Bovine Serum (Sigma F0926, Sigma Aldrich Corp., St. Louis, Mo., USA). The DLD-1, SW480 and SW620 colon cancer cells were cultured in DMEM (Sigma D6429) containing 10% (v/v) Fetal Bovine Serum (Sigma, F0926). For proliferation assays, cells (3.5×10⁴ cells per well) were split into 12-well plates. After 24 h, 1 μL of each compound in DMSO solution were added to each well. DMSO was used as a control. Each experiment was done in triplicate. Cell viability and number were analyzed using the Vi-Cell XR Cell Viability Analyzer (Beckman Coulter, Indianapolis, Ind., USA).

Biochemistry. Western blotting: Cells were lysed in the appropriate volume of lysis buffer: 50 mM HEPES, 100 mM NaCl, 2 mM EDTA, 1% (v/v) glycerol, 50 mM NaF, 1 mM Na₃VO₄, 1% (v/v) Triton X-100, with protease inhibitors. The following antibodies were used: AMPK (Cell Signaling, 2532, Cell Signaling Technologies, Danver, Mass., USA), pAMPK (Cell Signaling, 2535), ACC (Cell Signaling, 3676), pACC (Cell Signaling, 11818), Actin (Sigma, A1978). Antibodies for Axin 2, c-Myc and Cyclin D1 have been described previously. ATP analysis: Cells growing in 12-well plates were treated with DMSO or inhibitors in DMSO solution and lysed by adding 1 mL boiling doubly distilled water. Supernatants were analyzed by luminescence using ATP Determination Kit (Invitrogen, A22066; Thermo Fisher Scientific, Waltham, Mass., USA).

Reporter assay. Wnt reporter assay and cell staining assay have been described previously. We subcloned Super 8×TOPFlash (provided by Professor Randall Moon, University of Washington) into the pGL4.83 [hRlucP/Puro] Vector and transfected it into HEK293T cells. A stable HEK293T cell line containing the TOPFlash reporter was established using puromycin selection. To screen Wnt inhibitors that block the downstream signaling transduction pathway of β-catenin, we treated the reporter cells with 25 mM LiCl to stabilize β-catenin and activate Wnt signaling. The potential Wnt inhibitors identified from screening were validated by transfecting TOPFlash or FOPFlash plus control renilla reporters into HEK293T cells, and then treating the cells with DMSO or testing compounds in DMSO solution. The Wnt signaling was activated by LiCl or Wnt3A treatment.

Mitochondria Membrane potential assay: DLD-1 and LS174T cells were plated onto 24-well plates and maintained in DMEM (DLD-1) and RPMI1640 (LS174T) with 10% (v/v) (DLD-1) and 5% (v/v) (LS174T) serum for one day. The cells were treated with DMSO or a testing compound in DMSO solution for 2 h, followed by staining with 100 nM tetramethylrhodamine methyl ester perchlorate (TMRM) (Cayman Chemical Company, MI, USA) for 15 min. Cells were rinsed once with PBS and then observed under fluorescence microscopy at 568 nm.

Seahorse assay. Approximately 3×10⁴ cells were seeded in XF96 Cell Culture microplate (80 μL of 3.75×10⁵ cells/mL) for all experiments. On the next day, cell culture media were replaced with either Seahorse XF modified media with 25 mM glucose and 1 mM pyruvate. After media exchange, cells were treated with 1 μM of oligomycin A, 1.0 μM FCCP and mixture of 1.0 μM of rotenone and 1.0 μM of antimycin A in standard mitochondrial stress test conditions. To determine the uncoupler effects, FCCP was replaced with an equal volume of DMSO, or a compound to be tested in DMSO solution. ATP production rate was analyzed using Agilent Seahorse XF Real-Time ATP Rate Assay.

Isolation of Mitochondria from Brain Tissue. A mitochondrial isolation protocol was adapted from the previously described protocols with slight modifications. All the steps were carried out at 4° C. or on ice. After mice were euthanized with CO₂ followed by rapid decapitation, whole liver was quickly dissected out on a cold block and homogenized using a Teflon-glass dounce homogenizer containing isolation buffer (215 mM mannitol, 75 mM sucrose, 0.1% BSA, 20 mM HEPES, 1 mM EGTA, adjusted to pH 7.2 with KOH). The homogenate was transferred to a 2 mL-microcentrifuge tube and spun at 1,300×g for 3 min. The supernatant was transferred to a fresh 2 mL-microcentrifuge tube and spun at 13,000×g for 10 min. The supernatant was discarded, and the crude mitochondrial pellet was resuspended in a 1.5 mL-microcentrifuge tubes and pelleted again at 10,000×g for 10 min. The supernatant was discarded, and the mitochondrial pellets were resuspended in isolation buffer to obtain an approximate concentration ≥10 mg/mL of mitochondria. The absolute protein concentration was determined using a bicinchoninic acid (BCA) protein assay kit (Pierce, Cat #23,227) by recording absorbance at 560 nm on a Biotek Synergy HT plate reader (Winooski, Vt., USA).

Mitochondrial Bioenergetics Measurements. Mitochondrial bioenergetic measurements were carried out using a Seahorse XFe96 Extracellular Flux Analyzer (Agilent Technologies, Santa Clara, Calif., USA) to measure oxygen consumption rate (OCR) during various states of respiration. The OCR were measured in the presence of different substrates, inhibitors and uncouplers of the electron transport chain using previous methods with slight modifications. The stocks used for the assays were 500 mM pyruvate, 250 mM malate, and 30 mM adenosine diphosphate (ADP), and 1 M succinate (pH for all was adjusted to 7.2). Stock assay solutions of 1 mg/mL (1.26 mM) oligomycin A, 1 mM N-(4-(trifluoromethoxy)phenyl)carbonohydrazonoyl dicyanide (FCCP), and 1 mM rotenone were prepared in ethanol. Stock solutions of FH535, Y3, FH535-M, and Y3-M were prepared at 5 mM in 100% DMSO. As per the instructions from XFe96 Extracellular Flux kit, the sensor cartridge was hydrated with water and kept at 37° C. overnight before the experiment. One hour before the assay was conducted, water was removed from assay plates and XF calibrant was added before re-incubation. The injection ports A to D of the sensor cartridge were loaded with 25 μL of different combinations of the above substrates/inhibitors/uncouplers as follows. Before loading, the stocks were diluted appropriately in the respiration buffer (RB) (125 mM KCl, 0.1% BSA, 20 mM HEPES, 2 mM MgCl₂, and 2.5 mM KH₂PO₄; pH 7.2) to get the final concentrations in the respiration chamber of 5 μM pyruvate, 2.5 μM malate, 10 μM of succinate and 1 μM ADP (via Port A), 1 μM oligomycin A (via Port B), 4 μM FCCP or 5 μM/1 μM FH535 or 5 μM/1 μM Y3 or 5 μM/1 μM FH535-M or 5 μM/1 μM Y3-M (via Port C) and 0.1 μM antimycin A (via Port D) starting with the initial volume of 175 μL RB in the chamber and diluting it to 9×, 10×, 11× and 12× with every injection through ports A to D, respectively. Once loaded, the sensor cartridge was placed into the Seahorse XFe96 Flux Analyzer for automated calibration. Seahorse Standard XFe96 assay plates were used for loading mitochondria. Initially total mitochondria were diluted to 6 μg/30 μL in RB and 30 μL was loaded in each well resulting in 6 μg mitochondria/well. The assay plates were centrifuged at 3,000 rpm for 4 min at 4° C. to adhere liver mitochondria at the bottom of the wells. After centrifugation, 145 μL RB (pre-incubated to 37° C.) was added without disturbing the mitochondrial layer to obtain a final volume of 175 μL per well. After the instrument calibration with the sensor cartridge was complete, the utility plate was replaced by the plate loaded with mitochondria for bioenergetics analysis. The assays were carried out under a previously optimized protocol. Briefly, it involved cyclic steps of mixing, sequential injections of substrates/inhibitors via Ports A thru D, mixing, equilibration, and measurement of the OCR through fluorimetric optical probes. The data output gives State III respiration mediated through complex I and II in the presence of pyruvate, malate (PM), succinate and ADP (Port A) followed by State IV rate in presence of oligomycin (Port B). OCR was then measured in the presence of FCCP or compounds of interest in this study to examine mitochondrial specific uncoupling activity State V_{CI+CII} (Port C) and finally non-mitochondrial respiration in presence of Antimycin A (Port D).

Statistics. All experiments were performed with at least three independent repeats. Statistical analysis was performed using GraphPad Prism 7 (GraphPad Software, CA, USA). For all analyses, the significance of differences among groups was set at p<0.05. Error bars represent standard deviations. The Seahorse results were analyzed with Seahorse Wave Desktop Software.

### Example 2—Autophagic Flux Modulation by Wnt/β-catenin Pathway Inhibition in Hepatocellular Carcinoma

Abstract

Autophagy targets cellular components for lysosomal-dependent degradation in which the products of degradation may be recycled for protein synthesis and utilized for energy production. Autophagy also plays a critical role in cell homeostasis and the regulation of many physiological and pathological processes and prompts this investigation of new agents to effect abnormal autophagy in hepatocellular carcinoma (HCC). 2,5-Dichloro-N-(2-methyl-4-nitrophenyl)benzenesulfonamide (FH535) is a synthetic inhibitor of the Wnt/β-catenin pathway that exhibits anti-proliferative and anti-angiogenic effects on different types of cancer cells. The combination of FH535 with sorafenib promotes a synergistic inhibition of HCC and liver cancer stem cell proliferation, mediated in part by the simultaneous disruption of mitochondrial respiration and glycolysis. We demonstrated that FH535 decreased HCC tumor progression in a mouse xenograft model. For the first time, we showed the inhibitory effect of an FH535 derivative, FH535-N, alone and in combination with sorafenib on HCC cell proliferation. Our study revealed the contributing effect of Wnt/β-catenin pathway inhibition by FH535 and its derivative (FH535-N) through disruption of the autophagic flux in HCC cells.

Introduction

Hepatocellular carcinoma (HCC) is the most prevalent, primary malignancy of the liver and one of the leading causes of cancer-related deaths. Current statistics indicate this cancer affects over 700,000 people worldwide and causes an estimated 600,000 deaths annually. Despite improvements in prevention, early diagnosis and new treatments, the mortality of patients with HCC continues to rise and, over the past two decades, the incidence of HCC in the United States has tripled. The poor prognosis for patients with HCC reflects a pattern of the initial, undetected subclinical progression that ultimately results in late diagnosis when treatment options are limited. Current efforts are underway to find improved therapeutic strategies for HCC-related signaling pathways involved in the initiation and progression of tumors. Among these pathways, HCC displays altered Wnt/β-catenin signaling in which more than one-third of HCC cases exhibit cytoplasmic and/or nuclear accumulation of β-catenin, a finding that correlates with poor differentiation and prognosis. This pathway also contributes to the maintenance of tumor initiating cells, drug resistance and metastasis. There is increasing evidence for the interplay between the Wnt/β-catenin pathway and autophagy in different cancers. Autophagy is a highly conserved process that targets cellular components for lysosomal-dependent degradation in which the products of degradation may be recycled for protein synthesis and utilized for energy production. Autophagy also prevents accumulation of non-functional protein aggregates and organelles that are potentially damaging to the cell, and under stress-induced conditions might trigger tumor initiation. The fact that overactive autophagy can also support tumor development underscores the important role and tight regulatory requirements of this process in normal cell development and function. In this context, targeting the autophagy pathway emerges as a novel therapeutic opportunity for cancer treatment even if the regulation of this complex process and the involvement of different cell signaling pathways remain poorly understood. In HCC, a multi-kinase inhibitor, sorafenib, which is the standard treatment for advanced HCC, reportedly enhances autophagy. Chloroquine (CQ), an autophagy inhibitor, sensitizes HCC cells to the antineoplastic effects of sorafenib. This finding again suggests that modulation of autophagy represents a potential therapeutic target for HCC.

2,5-Dichloro-N-(2-methyl-4-nitrophenyl)benzenesulfonamide (FH535) is a synthetic inhibitor of the Wnt/β-catenin pathway that exhibits anti-proliferative and anti-angiogenic effects on different types of cancer cells. In previous studies, the combination of FH535 with sorafenib promoted a synergistic inhibition of HCC and liver cancer stem cell proliferation, mediated in part by the simultaneous disruption of mitochondrial respiration and glycolysis. In this study, we investigate the effect of FH535 on HCC tumor progression in a mouse xenograft model and explore the underlying mechanism of FH535 and its derivatives in modulating the Wnt/0-catenin-dependent autophagic flux in HCC.

Materials and Methods

Cell Lines

The HCC cell line Huh7 was a gift from Dr. Guangxiang Luo (University of Alabama, Birmingham). The HCC cell lines Hep3B and PLC were purchased from American Type Culture Collection (ATCC; Manassas, Va., USA). These cell lines were cultured in Dulbecco's Modified Eagles Medium (DMEM; Gibco, USA) supplemented with 10% fetal bovine serum (FBS; Gibco, USA), non-essential aminoacids (NEAA; Gibco, USA) and penicillin/streptomycin (Gibco, USA) and maintained in a NuAire incubator (Plymouth, Mich., USA) at 37° C. with 5% CO₂.

Animal Xenograft Model

All animal experiments were performed in accord with the guidelines, rules and recommendations and was approved by the University of Kentucky Institutional Animal Care and Use Committee after approval (IACUC). Three- to four-weeks old female athymic nude mice (nu/nu, The Jackson Laboratory, USA) were housed in a pathogen-free environment at the Division of Laboratory Animal Resources of the Chandler Medical Center, University of Kentucky. To generate in vivo tumors, Huh7 culture cells in mid-log phase growth were collected and re-suspended in a 50% mixture of Matrigel (BD Biosciences, USA) in serum-free medium to a final concentration of 6×10⁷ cells per mL. A volume of 0.1 mL of the cell suspension was injected subcutaneously in the right flank of each mouse. Mice were weighed and checked for tumor growth every other day. When tumors reached a volume of 100 mm³, mice were randomly divided into two groups of 5: vehicle control group and FH535 group (receiving 15 mg of FH535/kg/day from a stock prepared in dimethyl sulfoxide (DMSO) at 21.7 mg/mL and diluted in serum-free medium to a final concentration of 40% DMSO). Vehicle and FH535 were administered by intraperitoneal injection every other day. Tumors were measured using an optical caliper and tumor size was calculated using the formula: 0.5×length×(width)². Mice were euthanized at the end of the experiment or when reaching humane end-point following AVMA guidelines. Humane end-points included animals with tumors exceeding 20 mm in maximum diameter, with ulcerated tumors, more than 20% body weight loss, impaired mobility, labored breathing or with a body condition score below 2.

Hematoxylin and Eosin (H&E) and Immunohistochemistry of Explanted Tumors

Tumors from the xenograft model were formaldehyde fixed and paraffin-embedded and were used to performed H&E staining and immunohistochemistry of Ki-67 according to standard procedures.

Western Blot Analyses

Cell lysates were prepared in ice-cold RIPA buffer with freshly added protease inhibitor cocktail (ThermoFisher, USA). Protein concentration was determined using the BCA Protein Assay (ThermoFisher, USA). Cellular proteins (20-40m) were separated on SDS-polyacrylamide gel and transferred to PVDF membrane (ThermoFisher, USA). Primary antibodies are described in Table 1. All primary antibodies were used at 1:1000 dilution dilution with exception of the (3-actin antibody at 1:10000 following manufacturer recommendations. Proteins were detected by incubating with horseradish peroxidase-conjugated antibodies (Cell Signaling Technology, USA). Specific bands were visualized with enhanced chemiluminescence reagent (BioRad, USA) and quantified using the ImageJ software (Bethesda, Md., USA).

TABLE 1

| Antibody | Vendor | Catalog # |
|---|---|---|
| LC3B | Sigma | L7543 |
| P62 | cell signaling Technologies | 8025 |
| β-actin | Sigma | A5441 |
| β-catenin | cell signaling Technologies | 8480 |
| Survivin | cell signaling Technologies | 2808 |
| Cyclin D1 | cell signaling Technologies | 2978 |
| cMYC | cell signaling Technologies | 5605 |

Quantitative Real Time RT-PCR

Total RNA was extracted with miRNeasy mini kit (Qiagen, Germany), and the corresponding cDNA was produced using iScript cDNA synthesis kit (BioRad, USA) from 1 μg of total RNA. Real time quantitative PCR (RT-qPCR) was performed using SsoAdvanced Universal SYBR Green supermix (BioRad, USA) with specific primers: p62 (SQSTM1: 5′-AAGCCGGGTGGGAATGTTG-3′ (SEQ ID NO: 1) and 5′-GCTTGGCCCTTCGGATTCT-3′ (SEQ ID NO: 2)); c-MYC (cMYC: 5′-TTTTCGGGTAGTGGAAAACCAGC-3′ (SEQ ID NO: 3) and 5′-AGTAGAAATACGGCTGCACCGA-3′ (SEQ ID NO: 4)), Survivin (BIRC5, 5′-CAAGGAGCTGGAAGGCTGG-3′ (SEQ ID NO: 5) and 5′-GTTCTTGGCTCTTTCTCTGTCC-3′ (SEQ ID NO: 6)), AXIN 2 (AXIN2: 5′-CAGCAGAGGGACAGGAATCATT-3′ (SEQ ID NO: 7) and 5′-GCCAGTTTCTTTGGCTCTTTGTG-3′ (SEQ ID NO: 8)), Cyclin D1 (5′-CCND1: GGATGCTGGAGGTCTGCGA-3′ (SEQ ID NO: 9) and 5′-TAGAGGCCACGAACATGCAAGT-3′ (SEQ ID NO: 10)), and b2-microglobulin (B2M: 5′-GACTTTGTCACAGCCCAAGATAG-3′ (SEQ ID NO: 11) and 5′-TCCAATCCAAATGCGGCATCTTC-3′ (SEQ ID NO: 12)). Transcript levels were normalized to β-actin or β-2-macroglobulin level as indicated using the ΔΔCt method.

Metabolic Analysis

Oxygen Consumption Rates (OCR) were measured on an XF-96 Extracellular Flux Analyzer (Seahorse Bioscience) using the protocol and conditions optimized for HCC cells as previously described by our group. Briefly, the experiments were performed by seeding 10,000 and 20,000 Huh7 cells per well in XFe 96 well-plates 36 h before the experiment. Cells were treated for 24 h with FH535, FH535-N or vehicle control. In OCR experiments the media was supplemented with 25 mM Glucose and 1 mM Pyruvate just before the assay. After minimal incubation time (˜20-30 min in non-CO₂ 37° C. incubator) mitochondrial stress test was initiated. During the assay, 4 different drugs with the following final concentrations were injected to all of the 96 wells: 1) Port A—1 μM Oligomycin A, 2) Port B—1.5 μM Carbonyl cyanide 4-(trifluoromethoxy) phenylhydrazone (FCCP), 3) Port C—200 mM Etomoxir, and 4) Port D—mixture of 1 μM Rotenone and 1 μM Antimycin A. All reagents used in the Seahorse experiments were purchased from Sigma-Aldrich. Analyses of data were performed with Wave 2.2 software (Seahorse Bioscience), Excel (Microsoft Office 2013) and Prism 7.0 (GraphPad Software).

Autophagy Assay

Autophagy responses were monitored with the Cyto-ID autophagy detection reagent 2.0 (Enzo Life Sciences, USA) according to the manufacturer's instructions. Huh7 and PLC/PRF/5 cells were seeded in 12-well plates and treated the following day with drugs or DMSO vehicle control at the indicated concentrations. CQ was added to the corresponding wells to a final concentration of 50 μM 8 h prior harvesting. After 40 h of drug treatment, cells were collected and assessed for viability with Zombie Violet dye solution (BioLegend, USA) followed by Cyto-ID autophagy detection reagent staining. Flow cytometry analyses were performed in a BD LSRII at the Flow Cytometry and Cell Sorting Shared Resource Facility of the University of Kentucky Markey Cancer Center and data were analyzed with the FlowJo Software version 7.6.5 (Tree Star, USA). The autophagic flux was quantified by subtracting the Cyto-ID MFI value of the sample without CQ from the Cyto-ID MFI value of the sample with CQ for each condition according to the formula:

ΔLC3II=LC3II(+CQ)−LC3II(−CQ) (1)

Analysis of Cyto-ID MFI was performed on live cells (Zombie negative stained population).

β-Catenin Knockdown

Huh7 cells were transfected using lipofectamine RNAiMAX reagent (Invitrogen, USA) with Silencer Select Negative Control No. 1 siRNA (Ambion, USA) or β-catenin siRNA (s438, Ambion, USA) at a final concentration of 10 nM.

### 2,5-Dichloro-N-(4-nitronaphthalen-1-yl)benzenesulfonamide (FH535-N)

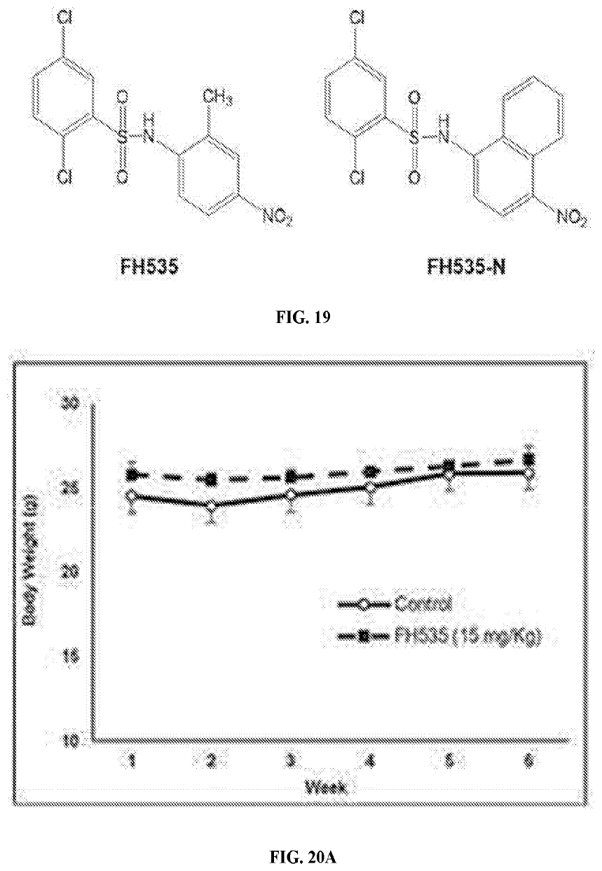

The synthesis of 2,5-dichloro-N-(4-nitronaphthalen-1-yl)benzenesulfonamide was described previously by Kril, et al., in which the N-(2-methyl-4-nitrophenyl) group of FH535 was replaced with a N-(4-nitronaphthyl) group (FIG. 19).

[³H]-Thymidine Incorporation Assay

Huh7, PLC/PRF/5 and Hep3B cells were plated in 96-well plates at 3000-4000 cells/well, treated with the concentrations indicated of FH535 or FH535-N, as single agents or in combination with sorafenib, and cultured for 72 h. ³H-thymidine incorporation assay was performed as described previously.

Apoptosis Assay

Apoptosis assay was performed in Huh7 and PLC/PRF/5 cells treated 48 h with DMSO vehicle control or the indicated doses of FH535, FH535-N alone or in combination with sorafenib. Cells were harvested and stained with the APC Annexin V apoptosis detection kit with PI (BioLegend, USA) according to the manufacturing instructions followed by flow cytometry analysis. Flow cytometry data was acquired with an LSRII instrument (BD-Biosciences) and analyzed with FlowJo software (Tree Star).

Dual Luciferase Assay

Cells were plated at 1×10⁵ cells/well in 24-well plates and transiently transfected with 490 ng of luciferase-reporter plasmid and 10 ng of phRL-TK per well using the Turbofect transfection reagent (Thermo Scientific, USA). After 5-6 h post-transfection, cells were treated with drug(s) or DMSO vehicle control for 36 h in the presence or absence of LiCl (10 mM). Luciferase assays were performed using the Dual Luciferase Assay System (Promega, USA) according to manufacturer's instructions.

Statistical Analysis

Data was reported as mean±SD of triplicate experiments (except where indicated). Statistical analyses were performed using GraphPad PRISM 7.0. Statistical significance of differences between two groups was analyzed using Student's t-test or ANOVA with post-hoc Tukey HSD accordingly. In all analyses, p<0.05 was considered a statistically significant difference.

Results

FH535 Inhibits the Growth of Xenograft Tumors in Mice

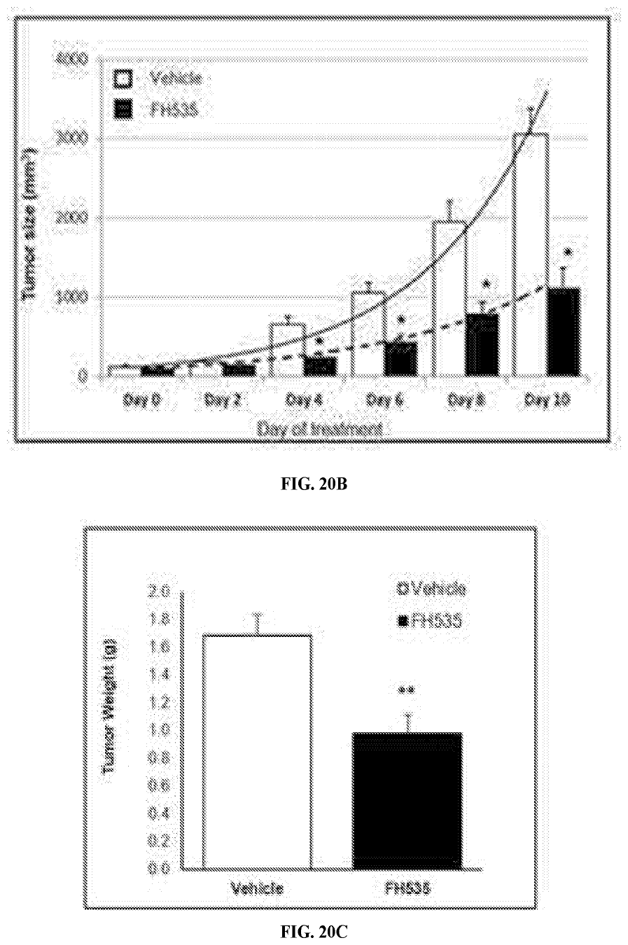

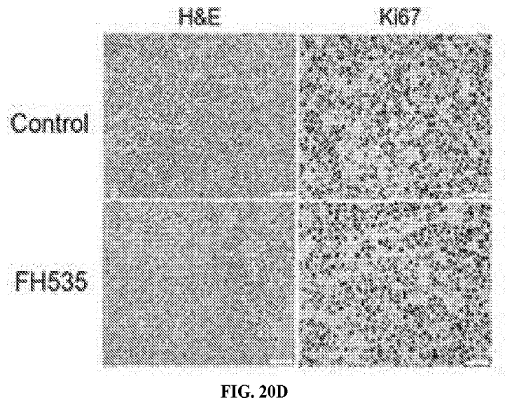

We previously showed that FH535 decreased the proliferation of different, human HCC cell lines, including Huh7 cells. To validate further the in vivo anti-tumor effect of FH535, we performed a gross-toxicity assay in mice with FH535 doses ranging from 0 to 30 mg/kg. We first demonstrated that intraperitoneal injections up to 15 mg/kg of FH535 for a period of 5-6 weeks did not induce major signs of body distress or toxicity such as weight loss, decreased ambulatory ability, labored respiration or dehydration (FIG. 20A). Next, we evaluated the in vivo anti-tumor activity of FH535 in a Huh7 tumor xenograft model. When HCC tumors reached a volume of 100 mm³, mice were injected with DMSO vehicle (control group) or 15 mg/kg of FH535 every other day. After only four days of treatment, the tumor volumes of FH535-treated mice were already significantly reduced compared to control group (p<0.05) (FIG. 20B-C). This result demonstrated the efficacy of the FH535 in vivo on the progression of HCC tumor growth. We also performed Hematoxylin and Eosin staining to assess tumor characteristics and showed that tumors in both groups were poorly differentiated HCC. We evaluated proliferation index using immunohistochemistry with Ki-67 expression, which demonstrated a proliferation index greater than 95% in both groups (FIG. 20D).

FH535 Affects Autophagy in HCC Cells

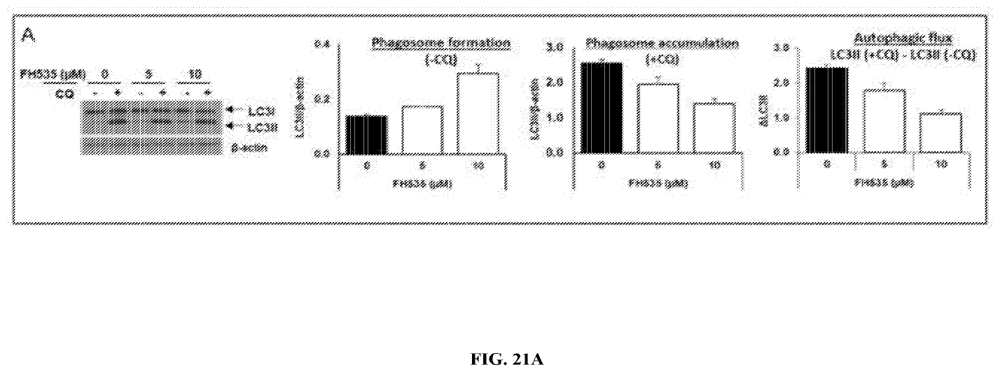

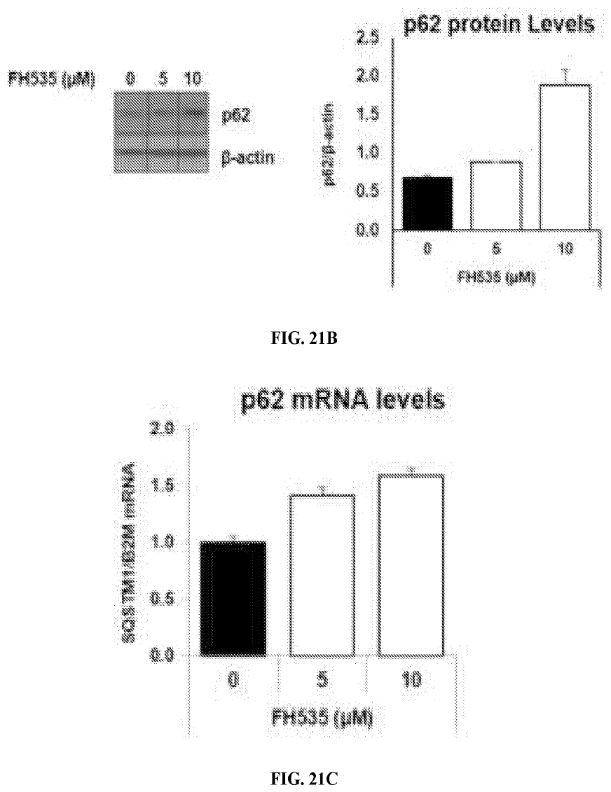

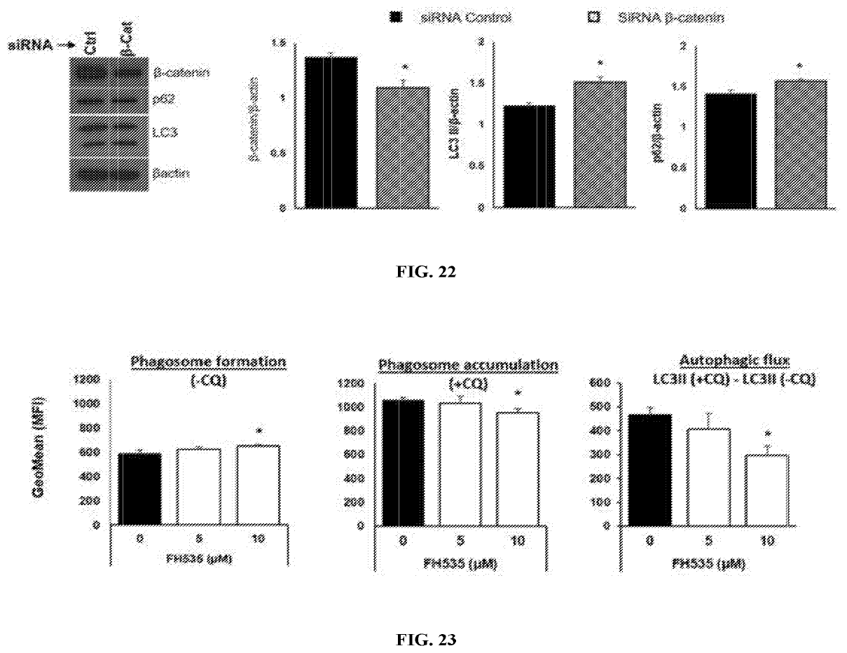

Increasing evidence for the crosstalk between Wnt/β-catenin and autophagy prompted an evaluation of the linkage between the anti-proliferative effect of FH535 on HCC and autophagy modulation. To investigate this possibility, we first examined LC3 expression levels as a marker of autophagic activity (FIG. 21A). Treatment of Huh7 cells with FH535 increased the lipid-bound expression of LC3II levels in a dose-dependent manner in comparison with control-treated cells, a finding that indicated an accumulation of autophagosomes (FIG. 21A, left panel). Consistent with the results observed for LC3II, western blot analyses indicated that p62, another autophagy marker, was also increased in FH535-treated cells (FIG. 21B). In addition, the knockdown of β-catenin in Huh7 cells increased the expression of both LC3II and p62, which is consistent with the targeting of β-catenin as potential mechanism of action of FH535 (FIG. 22). In this context, β-catenin was reported as a transcriptional repressor of p62, and as expected, our results verified the increase of p62 mRNA levels in the presence of FH535 (FIGS. 21B-C).

FH535 Modulates Autophagy Flux in HCC Cells

The accumulation of LC3II and p62 observed in FH535-treated cells were consistent with an effect on autophagy. This effect was attributed to changes in either autophagosome formation, the autophagic flux, or both. To discriminate among these processes, we analyzed the induced accumulation of LC3II protein levels after blocking the autophagosome degradation with CQ. As expected, the addition of CQ enhanced LC3II accumulation in both control and drug-treated cells compared to the corresponding experiments without CQ (FIG. 21A, middle panel). However, the progressive increase of LC3II levels in FH535-treated cells alone (FIG. 21A, first and second panels) and the reduced accumulation of LC3II in the presence of CQ (first, third and fourth panels) reflected the alteration of the autophagic activity in FH535-treated cells. To further support these findings, the Cyto-ID autophagy detection assay revealed a reduced accumulation of autophagic vesicles in response to FH535 treatment after addition of CQ (FIG. 23). Together, these results are consistent with the reduced autophagic flux in FH535-treated cells.

FH535-N on HCC Proliferation, Apoptosis and β-Catenin Pathway

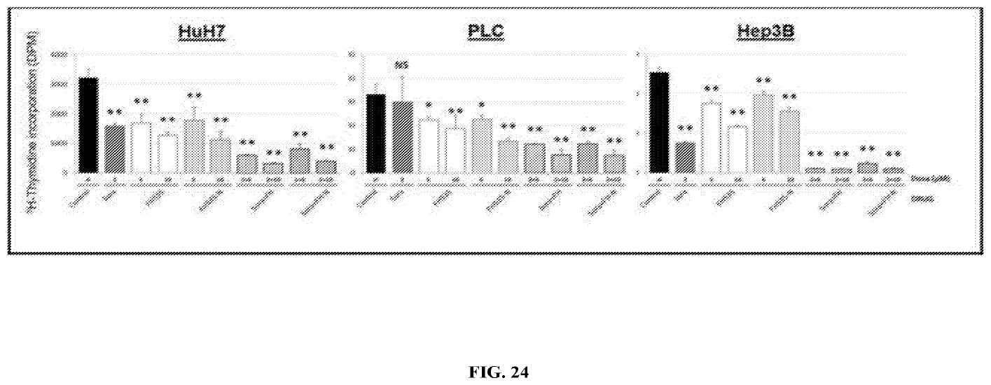

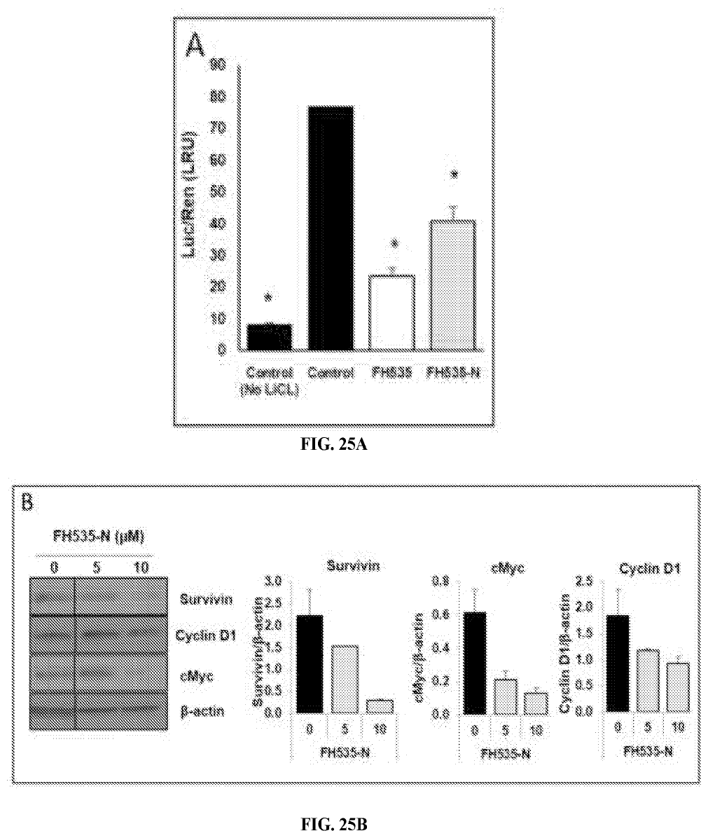

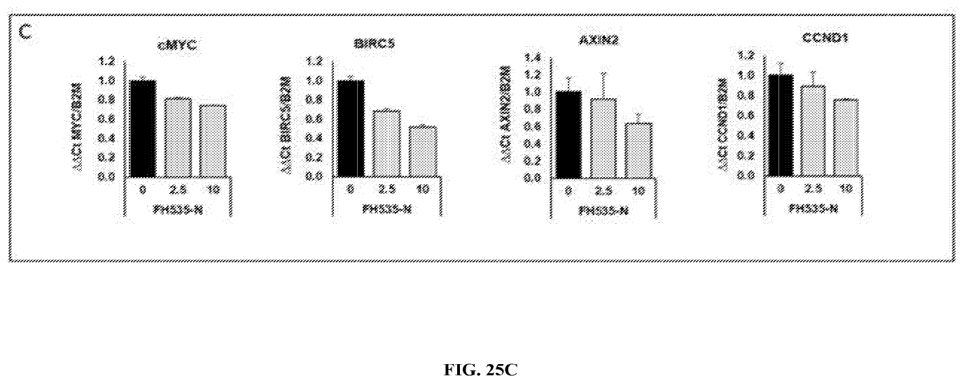

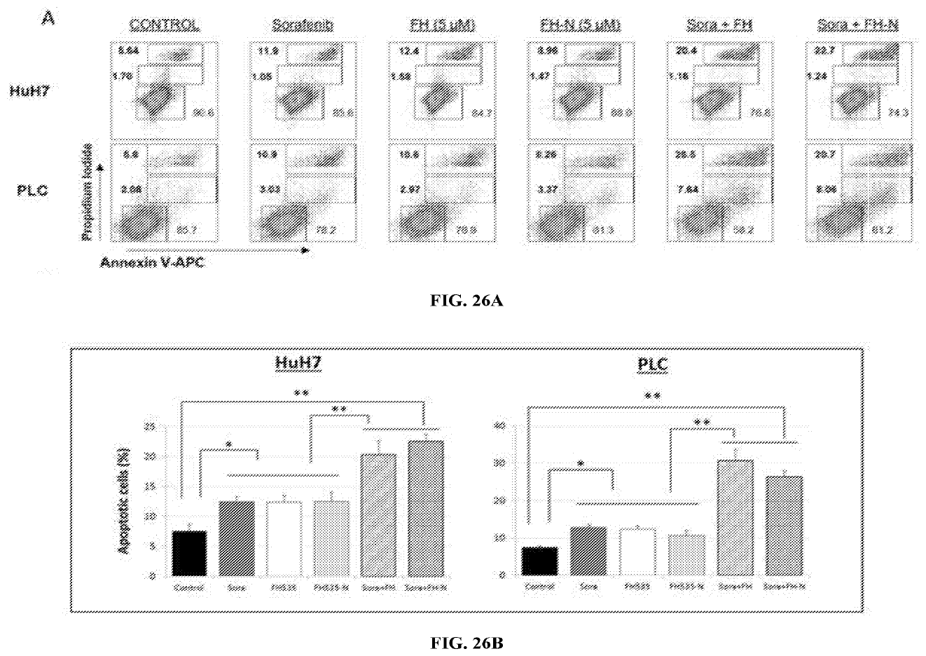

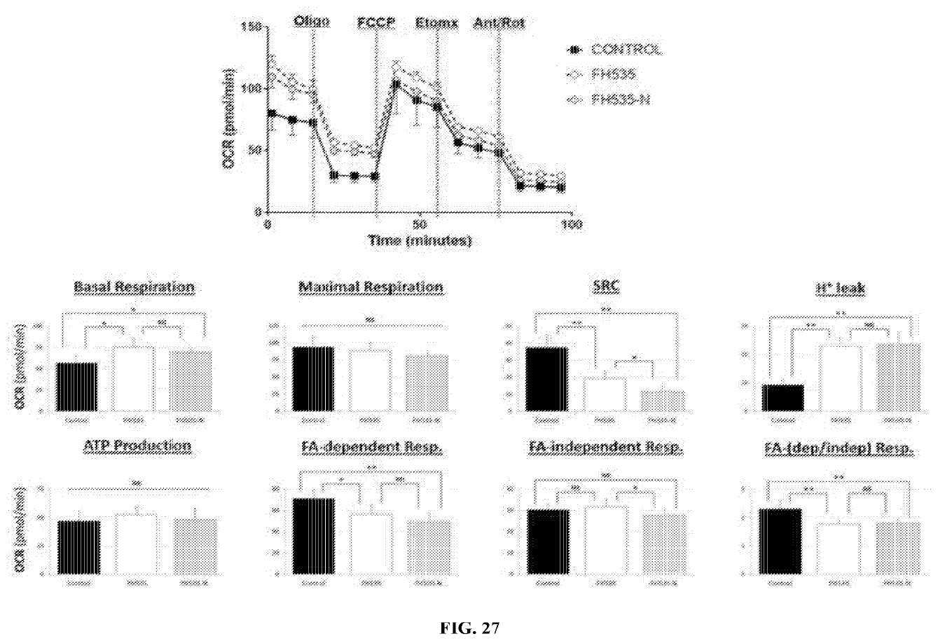

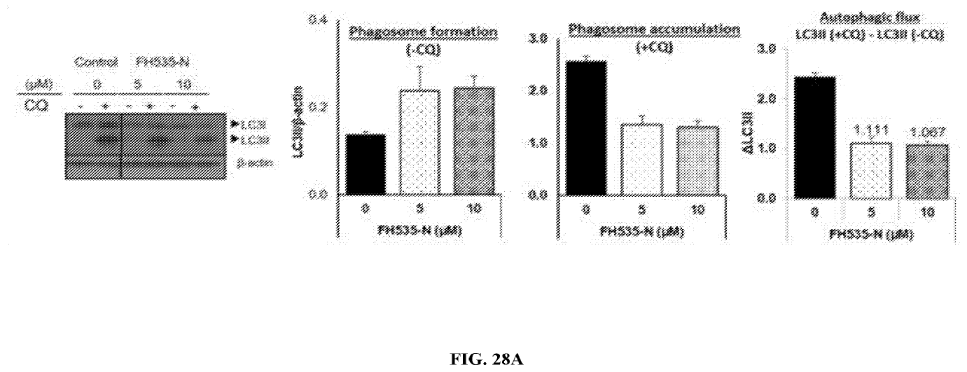

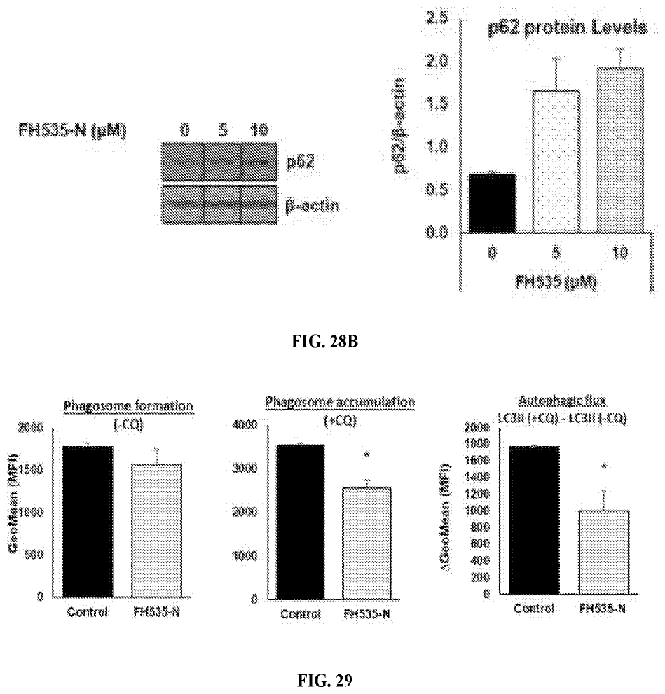

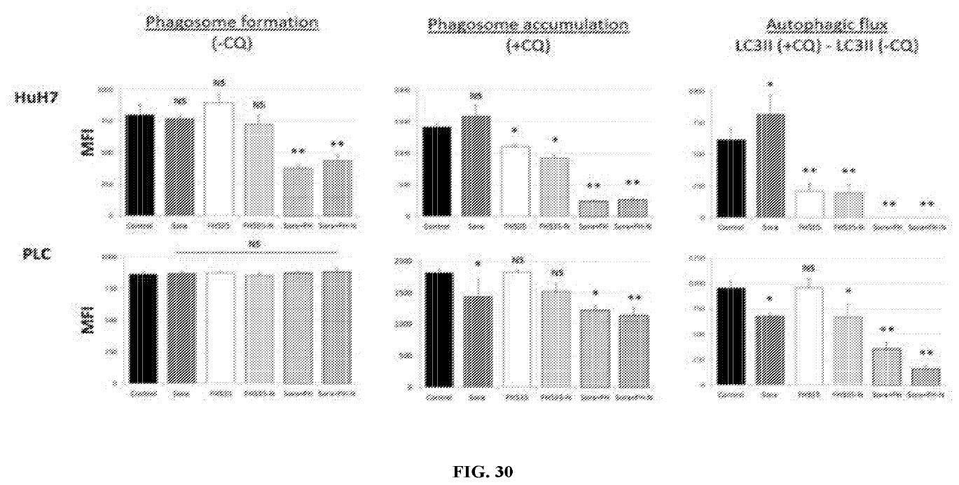

We previously described the synthesis of FH535 derivatives from commercially available halogen-substituted aryl sulfonyl chlorides and aryl amines. 2,5-Dichloro-N-(4-nitronaphthalen-1-yl)benzenesulfonamide (FH535-N) inhibited cell proliferation of Huh7, PLC and Hep3B cells (FIG. 24) and reduced the Wnt/β-catenin transcriptional activity as demonstrated by using a TOP-Flash TCF4-dependent luciferase reporter assay (FIG. 25A) as well as the expression of known downstream Wnt/β-catenin targets genes (FIGS. 25B-C). FH535-N demonstrated significant increased rate of apoptosis in Huh7 and PLC/PRF/5 (FIG. 26). In a previous article our group demonstrated the targeting of FH535 on mitochondrial respiration activity. Based on these results we performed a comparative study of the effects of FH535 and the derivative FH535-N on OCR. Our results, showed that both drugs induced similar inhibition of Spare Respiratory Capacity (SRC) and enhanced Proton Leak. These findings indicate similar alteration of the metabolic plasticity and increased oxidative stress of HCC cells treated with FH535 or FH535-N(FIG. 27). We analyzed the expression levels of LC3II and p62 in Huh7 cells after treatment with FH535-N in the presence and absence of CQ. Similar to the results observed with FH535, FH535-N also increased LC3II and p62 protein levels (FIGS. 28A-B). Addition of CQ demonstrated accumulation of LC3II with FH535-N and CQ treatment, as expected. However, this accumulation in the combination treatment is reduced at higher doses of FH535-N consistent with the results of FH535-treated cells. (FIG. 28A right panel). These results were consistent with a reduction in the autophagic flux in response to FH535-N in a fashion that mirrored the effects described for FH535. In addition, this possibility was supported by the results of FH535-N in Cyto-ID autophagy readouts (FIG. 29). Similar results were observed in PLC/PRF/5 cells (FIG. 30). Overall, our data demonstrated that the anti-proliferative effects of FH535 and its derivative, FH535-N, on HCC cells are associated with the regulation of autophagic processes.

FH535 and FH535-N in Combination with Sorafenib on HCC Cell Proliferation, Apoptosis and Autophagy

Our group have previously demonstrated a synergistic effect on cell proliferation using FH535 in combination with sorafenib. Due to these findings, we assessed the effect of drug combination of FH535 or FH535-N with sorafenib on HCC cell proliferation, apoptosis and autophagic flux. We found that FH535 and FH535-N have an additive effect on HCC cell proliferation of Huh7, PLC/PRF/5 and Hep3B in combination with sorafenib (FIG. 24). We have also observed a significant increase in apoptosis measured by Annexin V/PI using the combination treatments (FH535/sorafenib, FH535-N/sorafenib). This effect of the drug combination was more pronounced that the one seen with FH535, FH535-N or sorafenib alone (FIG. 26). FH535/sorafenib and FH535-N/sorafenib drug combination produced a significant decreased in the authophagic flux measured by CytoID (FIG. 29). We also used HCC cell lines Huh7 and PLC to perform WB assay to assess changes in P62 and LC3 expression with monotherapy and combination treatment. We found an accumulation of P62 and LC3-II in the presence of drug combination (FH535/sorafenib and FH535-N/sorafenib).

Discussion

Aberrant activation of the Wnt/β-catenin pathway occurs in numerous malignancies, including HCC. The poor prognosis and disease progression in liver cancer typically involves the upregulation of the Wnt/β-catenin pathway, and recent efforts focus on the development of new compounds targeting this and other signaling pathways as effective therapeutic alternatives for advanced HCC. The N-aryl benezenesulfonamides, such as FH535, inhibits the Wnt/β-catenin signaling pathway and the PPARs δ and γ with demonstrated anti-proliferative effect against pancreatic cancer, breast cancer, colorectal carcinoma and HCC cells. FH535 also sensitizes and reverses the epithelial-mesenchymal transition phenotype of radio-resistant esophageal cancer cells. In vivo, FH535 effectively suppresses growth and angiogenesis in pancreatic cancer and decreases tumor burden and progression in colorectal cancer. We now demonstrate the potent effects of FH535 on HCC tumor progression in vivo using a mouse xenograft model while showing no significant drug toxicity in the host.

Although, there is important pre-clinical evidence for the anti-cancer effects of FH535, the mechanism of action of this drug remains poorly understood. We recently demonstrated that FH535 induces changes in mitochondrial membrane potential and overall mitochondrial health in HCC tumor cells. FH535 targets specifically the electron transport chain complexes I and II and results in defective mitochondrial respiration. Since mitochondrial dysfunction and Wnt/β-catenin signaling affect the regulation of the autophagy process, this study reports on the anti-tumor effect of FH535 and its derivative FH535-N on HCC cells through the modulation of the autophagic activity.

Compared to untreated-control cells, our results demonstrate that FH535 increased LC3II and p62 levels in HCC cells, a finding that is indicative of autophagosomal accumulation by the increase in autophagosome formation and/or by a defective lysosomal degradative machinery. A modest CQ-induced increase in autophagosome accumulation occurs in FH535-treated cells, together with the reduced ΔLC3II levels in western blots, an additional finding that is consistent with an impaired autophagic flux. This may contribute to the accumulation of dysfunctional mitochondria and account for the increased apoptosis in FH535-treated cells. Future studies will assess the involvement of FH535 on autophagosomal formation on HCC cells as suggested by the enhancement of p62/SQSTM1 gene expression.

Several studies reveal the complex interplay between Wnt/β-catenin signaling and autophagy. The coordinated regulation of Wnt/β-catenin signaling and autophagy processes occurs in different types of cancers, including the cytotoxic effect of resveratrol on breast cancer cells and the reduced gemcitabine-induced apoptosis in human osteosarcoma cells. In this regard, the inhibition of Wnt/β-catenin pathway induces the accumulation of autophagic proteins such as LC3-II, ATG7, Beclin-1 and p62 proteins. Reciprocally, induction of autophagy regulates the Wnt/β-catenin pathway by targeting the clearance of β-catenin and other proteins involved in Wnt signaling such as the Dishevelled protein. In agreement with these studies, our results show that FH535 treatment induces the accumulation of LC3II and p62 proteins as well as p62/SQSTM1 mRNA and suggests that the effect of FH535 on autophagy links to the inhibition of Wnt/β-catenin signaling. In support of this possibility, the β-catenin knockdown in HCC cells also exhibits a subsequent increase in LC3II and p62 protein levels.

N-Aryl benezenesulfonamides, such as FH535 and FH535-N exhibit significant anti-cancer effects. In this study, FH535 and FH535-N produce similar anti-proliferative activity in HCC cells. Furthermore, both compounds target the Wnt/β-catenin signaling pathway as indicated by β-catenin-dependent reporter assays (FIG. 25A) as well as the reduced expression of endogenous downstream β-catenin target genes (FIGS. 25B-C). Additionally, FH535-N induces the accumulation of autophagic proteins p62 and LC3II and impairs the autophagic flux in Huh? cells. These results provide further evidence that FH535-based derivatives warrant additional development as anti-cancer drug candidates for HCC treatment. Moreover, the synergistic effects of FH535 and sorafenib on the inhibition of HCC cell proliferation and survival was associated to the distinct targeting of both drugs on the mitochondrial function and metabolic pathways. Inhibition of autophagy by ATG7 knockdown or CQ treatment sensitizes HCC cells to sorafenib by enhancing apoptosis. Likewise, and consistent with these reports, our findings indicate that the inhibition of autophagic flux by FH535 and FH535-N contributes, at least partially, to the synergistic effects observed using FH535 in combination with sorafenib. We also demonstrate an additive effect of drug combination therapy using FH535 or FH535-N with sorafenib on HCC cell proliferation and apoptosis. Importantly, our findings revealed a significant increase in autophagic disruption caused by these combinatory treatments compared to FH535, FH535-N or sorafenib alone.

In conclusion, our data demonstrate potent anti-tumor effects of FH535 in vivo at dosage levels (15 mg/kg/day) that produce no gross toxicity in the mice. These studies also reveal a contributing mechanism for the anti-tumor action of FH535 with the Wnt/β-catenin-mediated regulation of the autophagy process. Further studies are warranted to assess the efficacy of FH535 and its derivatives either alone or in combination with conventional therapies as rational therapeutic alternatives for HCC treatment.

### Example 3

Novel Immunogenic Cell Death (ICD) Inducers for Cancer Therapy

Immune checkpoint blockage with monoclonal antibodies is a recent advance that provides new hope to cancer patients. Unfortunately, only limited cancer patients responded well to these antibodies. Compared with other cancers, patients with late-stage CRC responded very poorly to current immunotherapies. Only few cases of microsatellite instable CRC (account for 15% CRC) had response to immunotherapy. For this reason, therapeutic vaccines are being developed to be used in combination with antibodies for CRC treatment.

Recent improvements in immunotherapy have included the development of small-molecule immunogenic cell-death (ICD) inducers, which induce a special type of apoptosis. Most apoptotic cells are poorly immunogenic; however, some dying apoptotic cells release damage-associated molecular patterns (DAMPs) into the tumor microenvironment and these DAMPs stimulate an immune response. These dying cells also release tumor-specific antigens to attract immune cells. The chronic exposure of DAMPs in the tumor microenvironment that result in stimulating an anti-tumor immune response is defined as ICD. The tumor cells undergoing ICD produce “eat me signals” and thereby become a “tumor vaccine”. Since the discovery of ICD, several inducers have been identified, including bleomycin, cyclophosphamide, oxaliplatin, doxorubicin and some oncolytic peptide and virus. Other than the peptide and viral inducers, most of published ICD inducers are chemotherapeutic agents with adverse side-effects that also kill both cancer and normal cells, independent of their ICD effects. To improve on this situation, it will be important to develop a well-defined ICD inducer with a clear mechanism and minimal side-effects.

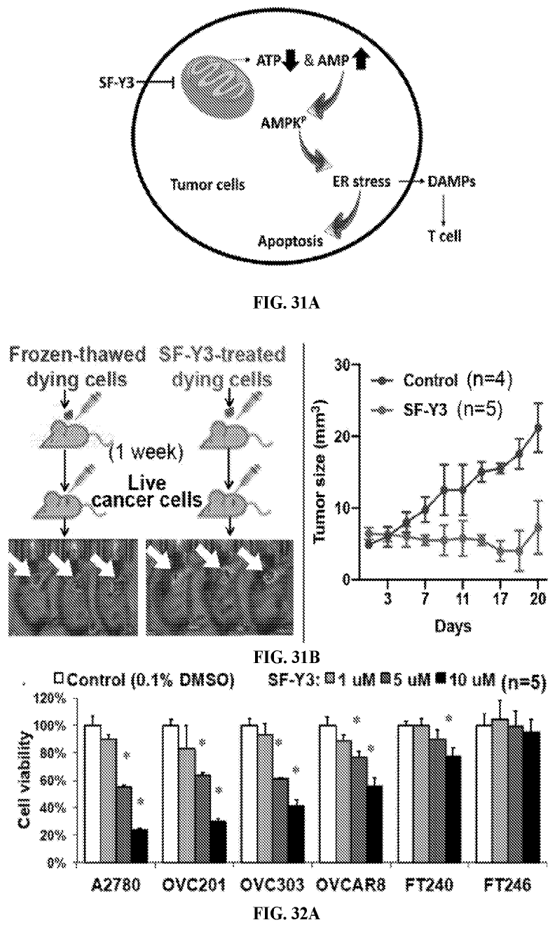

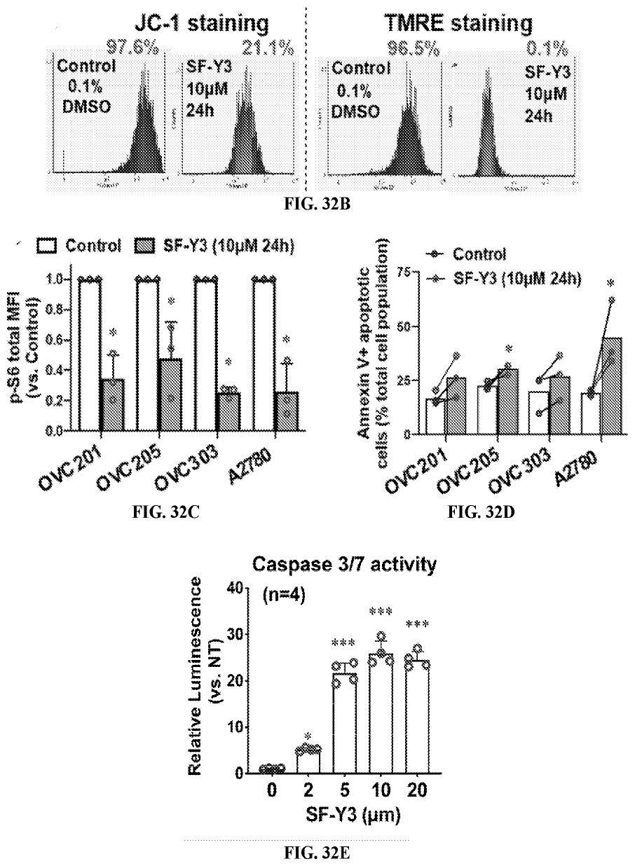

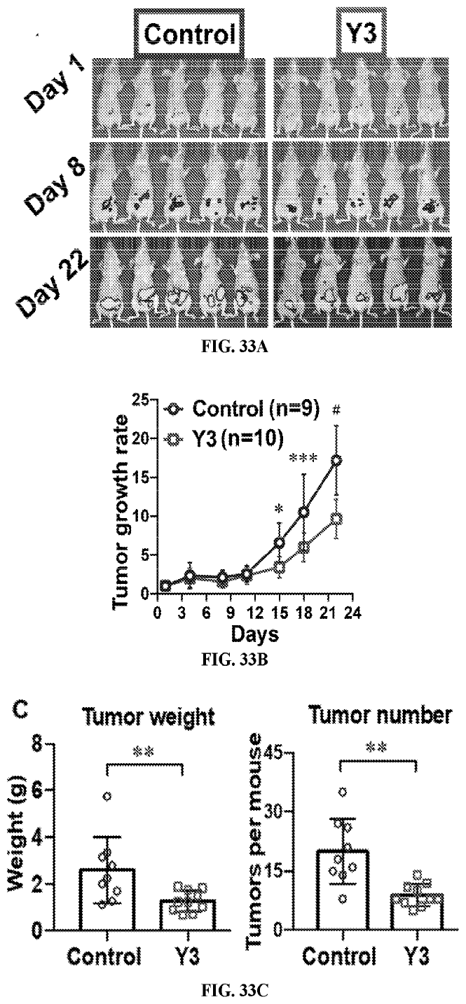

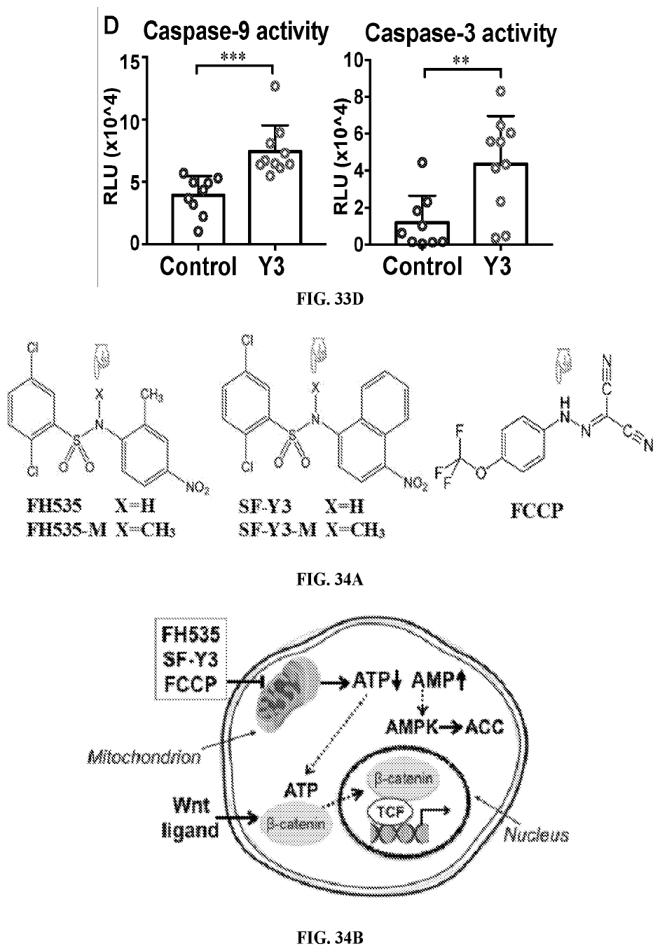

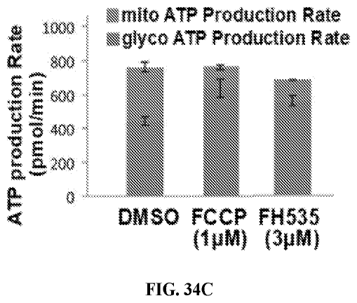

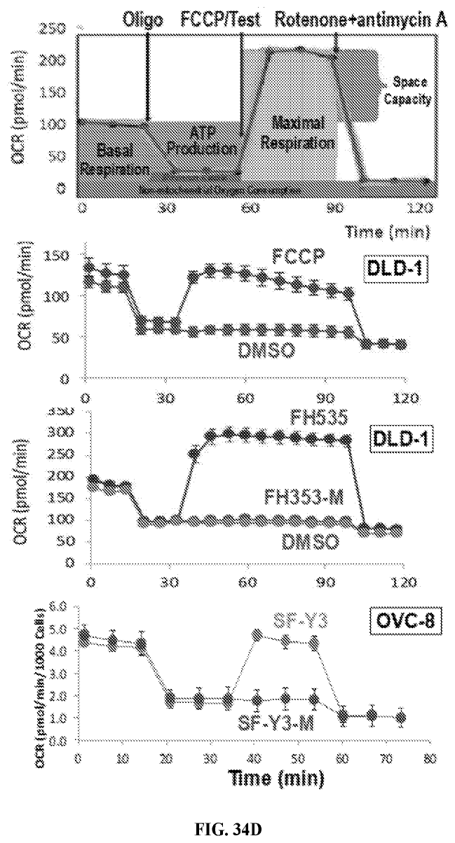

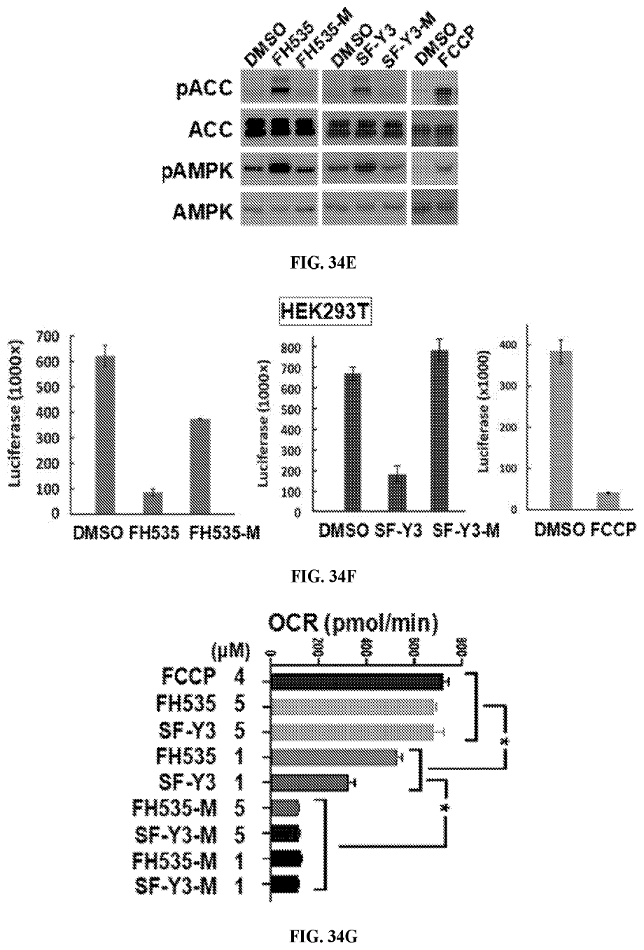

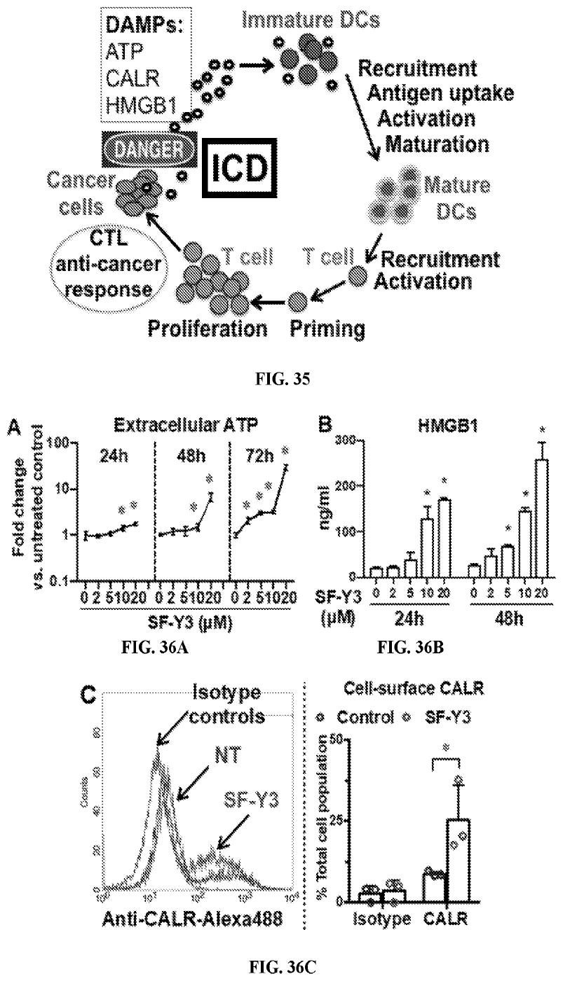

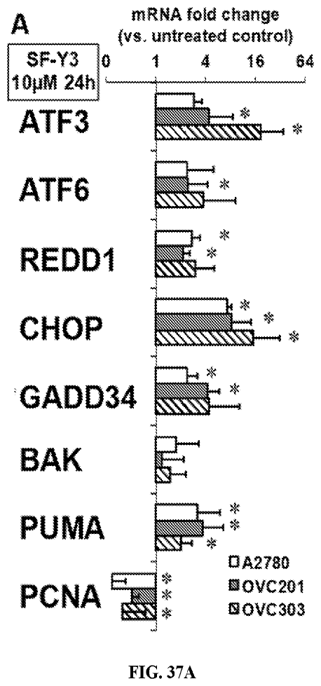

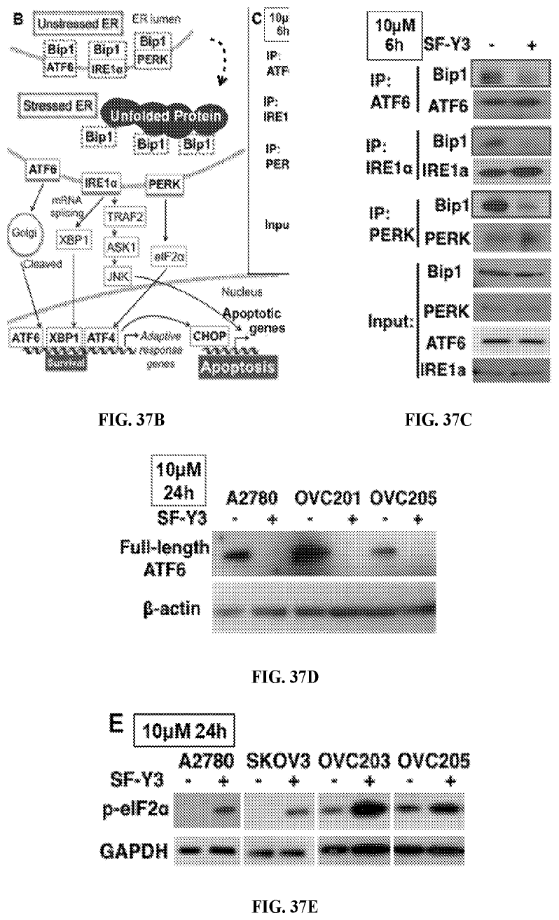

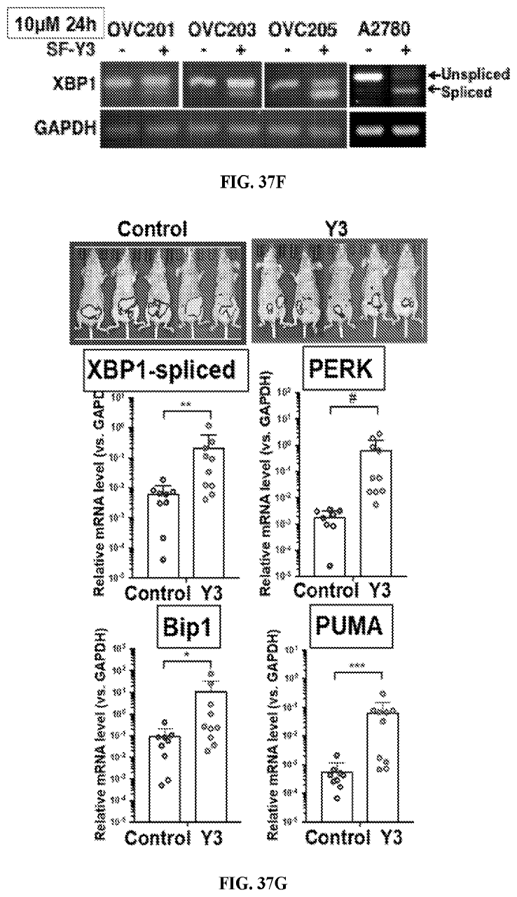

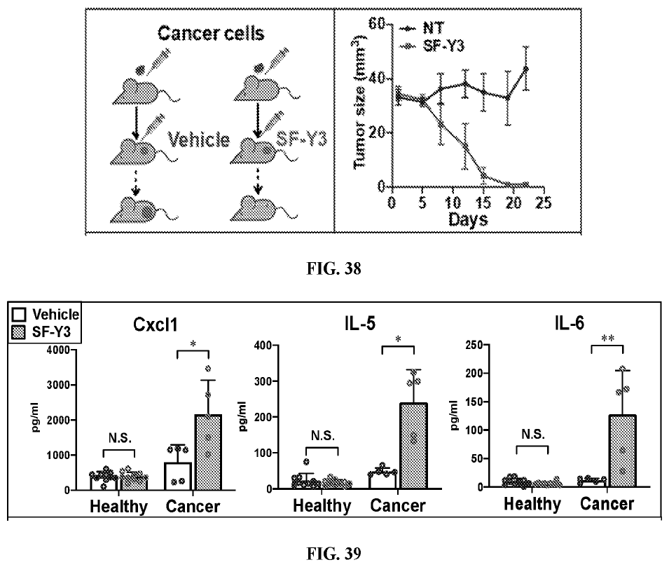

To address this unmet clinical need, we developed a family of novel ICD inducers that regulate mitochondrial function and ER stress (FIG. 31A). They were originally identified from a high-throughput screening as AMPK activators. Then, we found that they activated AMPK by acting as mitochondria uncouplers. Recently, we found that some of these uncouplers strongly induced ER stress (FIGS. 32A-39), which is a key factor for ICD. The gold-standard for ICD is the relocation and expose of ER chaperone calreticulin (CALR), secretion of ATP and release of HMGB1 from drug-treated tumor cells. One of our compounds, SF-Y3, induced all three DAMPs (FIGS. 32A-39). SF-Y3 inhibited tumor growth in both immunodeficient and immunocompetent mouse models of ovarian cancer (FIGS. 32A-39). In addition, SF-Y3 treated tumor cells can be used a tumor vaccine (FIG. 31B).

There are other ICD inducers developed from chemotherapy agents, oncolytic peptide and oncolytic virus. The technologies for peptide and virus are not as mature as small molecule ICD inducers. Most published small molecule ICD inducers are cytotoxic agents; they are equally toxic to normal cells and are not specific ICD inducers. Unlike some cytotoxic ICD inducers, which only induce one or two of these three DAMPs, our compounds induced all three DAMPs and can be used to generate tumor vaccine. These compounds can stimulate immune response by inducing cytokine secretion (FIGS. 32A-39). In addition, as mitochondrial uncouplers, these compounds can be used to treat multi-type of cancers and can be used in combination with immune checkpoint antibodies⁵.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Kadenbach, B. Intrinsic and extrinsic uncoupling of oxidative phosphorylation. Biochim Biophys Acta 2003, 1604, 77-94.
2. Childress, E. S.; Alexopoulos, S. J.; Hoehn, K. L.; Santos, W. L. Small Molecule Mitochondrial Uncouplers and Their Therapeutic Potential. J Med Chem 2018, 61, 4641-4655.
3. Hargreaves, I. P.; Al Shahrani, M.; Wainwright, L.; Heales, S. J. Drug-Induced Mitochondrial Toxicity. Drug Saf 2016, 39, 661-74.
4. Sviripa, V.; Zhang, W.; Conroy, M. D.; Schmidt, E. S.; Liu, A. X.; Truong, J.; Liu, C.; Watt, D. S. Fluorinated N,N′-diarylureas as AMPK activators. Bioorg Med Chem Lett 2013, 23, 1600-3.
5. Kril, L. M.; Vilchez, V.; Jiang, J.; Turcios, L.; Chen, C.; Sviripa, V. M.; Zhang, W.; Liu, C.; Spear, B.; Watt, D. S.; Gedaly, R. N-Aryl benzenesulfonamide inhibitors of [3H]-thymidine incorporation and beta-catenin signaling in human hepatocyte-derived Huh-7 carcinoma cells. Bioorg Med Chem Lett 2015, 25, 3897-9.
6. Chamoto, K.; Chowdhury, P. S.; Kumar, A.; Sonomura, K.; Matsuda, F.; Fagarasan, S.; Honjo, T. Mitochondrial activation chemicals synergize with surface receptor PD-1 blockade for T cell-dependent antitumor activity. Proc Natl Acad Sci USA 2017, 114, E761-E770.
7. Hubbard, W. B.; Harwood, C. L.; Geisler, J. G.; Vekaria, H. J.; Sullivan, P. G. Mitochondrial uncoupling prodrug improves tissue sparing, cognitive outcome, and mitochondrial bioenergetics after traumatic brain injury in male mice. J Neurosci Res 2018, 96, 1677-1688.
8. Figarola, J. L.; Weng, Y.; Lincoln, C.; Home, D.; Rahbar, S. Novel dichlorophenyl urea compounds inhibit proliferation of human leukemia HL-60 cells by inducing cell cycle arrest, differentiation and apoptosis. Invest New Drugs 2012, 30, 1413-25.
9. Farha, M. A.; Verschoor, C. P.; Bowdish, D.; Brown, E. D. Collapsing the proton motive force to identify synergistic combinations against Staphylococcus aureus. Chem Biol 2013, 20, 1168-78.
10. Goedeke, L.; Perry, R. J.; Shulman, G. I. Emerging Pharmacological Targets for the Treatment of Nonalcoholic Fatty Liver Disease, Insulin Resistance, and Type 2 Diabetes. Annu Rev Pharmacol Toxicol 2019, 59, 65-87.
11. Serviddio, G.; Bellanti, F.; Sastre, J.; Vendemiale, G.; Altomare, E. Targeting mitochondria: a new promising approach for the treatment of liver diseases. Curr Med Chem 2010, 17, 2325-37.
12. Ahmed K, Shaw H V, Koval A, Katanaev V L. A Second WNT for Old Drugs: Drug Repositioning against WNT-Dependent Cancers. Cancers (Basel). 2016; 8(7). doi: 10.3390/cancers8070066. PubMed PMID: 27429001; PubMed Central PMCID: PMCPMC4963 808.
13. Krishnamurthy N, Kurzrock R. Targeting the Wnt/beta-catenin pathway in cancer: Update on effectors and inhibitors. Cancer Treat Rev. 2018; 62:50-60. doi: 10.1016/j.ctrv.2017.11.002. PubMed PMID: 29169144; PubMed Central PMCID: PMCPMC5745276.
14. Nusse R, Clevers H. Wnt/beta-Catenin Signaling, Disease, and Emerging Therapeutic Modalities. Cell. 2017; 169(6):985-99. doi: 10.1016/j.cell.2017.05.016. PubMed PMID: 28575679.
15. Zimmerli D, Hausmann G, Cantu C, Basler K. Pharmacological interventions in the Wnt pathway: inhibition of Wnt secretion versus disrupting the protein-protein interfaces of nuclear factors. Br J Pharmacol. 2017; 174(24):4600-10. doi: 10.1111/bph.13864. PubMed PMID: 28521071; PubMed Central PMCID: PMCPMC5727313.
16. Shi, J.; Liu, Y.; Xu, X.; Zhang, W.; Yu, T.; Jia, J.; Liu, C. Deubiquitinase USP47/UBP64E Regulates beta-Catenin Ubiquitination and Degradation and Plays a Positive Role in Wnt Signaling. Mol Cell Biol 2015, 35, 3301-3311.
17. Kahn M. Can we safely target the WNT pathway? Nat Rev Drug Discov. 2014; 13(7):513-32. doi: 10.1038/nrd4233. PubMed PMID: 24981364; PubMed Central PMCID: PMCPMC4426976.
18. Liu C, Li Y, Semenov M, Han C, Baeg G H, Tan Y, et al. Control of beta-catenin phosphorylation/degradation by a dual-kinase mechanism. Cell. 2002; 108(6):837-47. PubMed PMID: 11955436.
19. Liu C, Kato Y, Zhang Z, Do V M, Yankner B A, He X. beta-Trcp couples beta-catenin phosphorylation-degradation and regulates Xenopus axis formation. Proc Natl Acad Sci USA. 1999; 96(11):6273-8. PubMed PMID: 10339577; PubMed Central PMCID: PMCPMC26871.
20. Liu C, He X. Destruction of a destructor: a new avenue for cancer therapeutics targeting the Wnt pathway. J Mol Cell Biol. 2010; 2(2):70-3. doi: 10.1093/jmcb/mjp040. PubMed PMID: 20008332; PubMed Central PMCID: PMCPMC2861491.
21. Chung, N.; Marine, S.; Smith, E. A.; Liehr, R.; Smith, S. T.; Locco, L.; Hudak, E.; Kreamer, A.; Rush, A.; Roberts, B.; Major, M. B.; Moon, R. T.; Arthur, W.; Cleary, M.; Strulovici, B.; Ferrer, M. A 1,536-well ultra-high-throughput siRNA screen to identify regulators of the Wnt/beta-catenin pathway. Assay Drug Dev Technol 2010, 8, 286-294.
22. James, R. G.; Davidson, K. C.; Bosch, K. A.; Biechele, T. L.; Robin, N. C.; Taylor, R. J.;

Major, M. B.; Camp, N. D.; Fowler, K.; Martins, T. J.; Moon, R. T. WIKI4, a novel inhibitor of tankyrase and Wnt/ss-catenin signaling. PLoS One 2012, 7, e50457.
23. Wang X, Moon J, Dodge M E, Pan X, Zhang L, Hanson J M, et al. The development of highly potent inhibitors for porcupine. J Med Chem. 2013; 56(6):2700-4. doi: 10.1021/jm400159c. PubMed PMID: 23477365; PubMed Central PMCID: PMCPMC3631274.
24. Huang S M, Mishina Y M, Liu S, Cheung A, Stegmeier F, Michaud G A, et al. Tankyrase inhibition stabilizes axin and antagonizes Wnt signalling. Nature. 2009; 461(7264):614-20. doi: 10.1038/nature08356. PubMed PMID: 19759537.
25. Emami K H, Nguyen C, Ma H, Kim D H, Jeong K W, Eguchi M, et al. A small molecule inhibitor of beta-catenin/CREB-binding protein transcription [corrected]. Proc Natl Acad Sci USA. 2004; 101(34):12682-7. doi: 10.1073/pnas.0404875101. PubMed PMID: 15314234; PubMed Central PMCID: PMCPMC515116.
26. Burikhanov, R.; Sviripa, V. M.; Hebbar, N.; Zhang, W.; Layton, W. J.; Hamza, A.; Zhan, C. G.; Watt, D. S.; Liu, C.; Rangnekar, V. M. Arylquins target vimentin to trigger Par-4 secretion for tumor cell apoptosis. Nat Chem Biol 2014, 10, 924-926.
27. Zhang, W.; Sviripa, V.; Chen, X.; Shi, J.; Yu, T.; Hamza, A.; Ward, N. D.; Kril, L. M.; Vander Kooi, C. W.; Zhan, C. G.; Evers, B. M.; Watt, D. S.; Liu, C. Fluorinated N,N-dialkylaminostilbenes repress colon cancer by targeting methionine S-adenosyltransferase 2A. ACS Chem Biol 2013, 8, 796-803.
28. Kenlan D E, Rychahou P, Sviripa V M, Weiss H L, Liu C, Watt D S, et al. Fluorinated N,N′-Diarylureas As Novel Therapeutic Agents Against Cancer Stem Cells. Mol Cancer Ther. 2017; 16(5):831-7. doi: 10.1158/1535-7163.MCT-15-0634. PubMed PMID: 28258165; PubMed Central PMCID: PMCPMC5418095.
29. Sviripa V, Zhang W, Conroy M D, Schmidt E S, Liu A X, Truong J, et al. Fluorinated N,N′-diarylureas as AMPK activators. Bioorg Med Chem Lett. 2013; 23(6):1600-3. doi: 10.1016/j.bmc1.2013.01.096. PubMed PMID: 23414799; PubMed Central PMCID: PMCPMC3594501.
30. Handeli S, Simon JA. A small-molecule inhibitor of Tcf/beta-catenin signaling down-regulates PPARgamma and PPARdelta activities. Mol Cancer Ther. 2008; 7(3):521-9. doi: 10.1158/1535-7163.MCT-07-2063. PubMed PMID: 18347139.
31. Zhou G, Myers R, Li Y, Chen Y, Shen X, Fenyk-Melody J, et al. Role of AMP-activated protein kinase in mechanism of metformin action. J Clin Invest. 2001; 108(8):1167-74. doi: 10.1172/JCI13505. PubMed PMID: 11602624; PubMed Central PMCID: PMCPMC209533.
32. Goransson, O.; McBride, A.; Hawley, S. A.; Ross, F. A.; Shpiro, N.; Foretz, M.; Viollet, B.; Hardie, D. G.; Sakamoto, K. Mechanism of action of A-769662, a valuable tool for activation of AMP-activated protein kinase. J Biol Chem 2007, 282, 32549-32560.
33. Hardie, D. G.; Ross, F. A.; Hawley, S. A. AMPK: a nutrient and energy sensor that maintains energy homeostasis. Nat Rev Mol Cell Biol 2012, 13, 251-262.
34. Kim, J.; Yang, G.; Kim, Y.; Kim, J.; Ha, J. AMPK activators: mechanisms of action and physiological activities. Exp Mol Med 2016, 48, e224.
35. Brand M D, Nicholls D G. Assessing mitochondrial dysfunction in cells. Biochem J. 2011; 435(2):297-312. doi: 10.1042/BJ20110162. PubMed PMID: 21726199; PubMed Central PMCID: PMCPMC3076726.
36. Turcios L, Vilchez V, Acosta L F, Poyil P, Butterfield D A, Mitov M, et al. Sorafenib and

FH535 in combination act synergistically on hepatocellular carcinoma by targeting cell bioenergetics and mitochondrial function. Dig Liver Dis. 2017; 49(6):697-704. doi: 10.1016/j.dld.2017.01.146. PubMed PMID: 28179093.
37. Bordwell, F. G. Equilibrium acidities in dimethyl sulfoxide solution. Acct. Chem. Res. 1988, 21, 456-463.
38. Porcelli, A. M.; Ghelli, A.; Zanna, C.; Pinton, P.; Rizzuto, R.; Rugolo, M. pH difference across the outer mitochondrial membrane measured with a green fluorescent protein mutant. Biochem Biophys Res Commun 2005, 326, 799-804.
39. Lou, P. H.; Hansen, B. S.; Olsen, P. H.; Tullin, S.; Murphy, M. P.; Brand, M. D. Mitochondrial uncouplers with an extraordinary dynamic range. Biochem J 2007, 407, 129-140.
40. Wick A N, Drury D R, Nakada H I, Wolfe J B. Localization of the primary metabolic block produced by 2-deoxyglucose. J Biol Chem. 1957; 224(2):963-9. PubMed PMID: 13405925.
41. Barker N, Hurlstone A, Musisi H, Miles A, Bienz M, Clevers H. The chromatin remodelling factor Brg-1 interacts with beta-catenin to promote target gene activation. EMBO J. 2001; 20(17):4935-43. doi: 10.1093/emboj/20.17.4935. PubMed PMID: 11532957; PubMed Central PMCID: PMCPMC125268.
42. Fagotto F, Gluck U, Gumbiner BM. Nuclear localization signal-independent and importin/karyopherin-independent nuclear import of beta-catenin. Curr Biol. 1998; 8(4):181-90. PubMed PMID: 9501980
43. Holik, A. Z.; Young, M.; Krzystyniak, J.; Williams, G. T.; Metzger, D.; Shorning, B. Y.; Clarke, A. R. Brg1 loss attenuates aberrant wnt-signalling and prevents wnt-dependent tumourigenesis in the murine small intestine. PLoS Genet 2014, 10, e1004453.
44. Costa, R.; Peruzzo, R.; Bachmann, M.; Monta, G. D.; Vicario, M.; Santinon, G.; Mattarei, A.; Moro, E.; Quintana-Cabrera, R.; Scorrano, L.; Zeviani, M.; Vallese, F.; Zoratti, M.; Paradisi, C.; Argenton, F.; Brini, M.; Cali, T.; Dupont, S.; Szabo, I.; Leanza, L. Impaired Mitochondrial ATP Production Downregulates Wnt Signaling via ER Stress Induction. Cell Rep 2019, 28, 1949-1960 e1946.
45. Brown, K.; Yang, P.; Salvador, D.; Kulikauskas, R.; Ruohola-Baker, H.; Robitaille, A. M.; Chien, A. J.; Moon, R. T.; Sherwood, V. WNT/beta-catenin signaling regulates mitochondrial activity to alter the oncogenic potential of melanoma in a PTEN-dependent manner. Oncogene 2017, 36, 3119-3136.
46. Kril, L. M.; Vilchez, V.; Jiang, J.; Turcios, L.; Chen, C.; Sviripa, V. M.; Zhang, W.; Liu, C.; Spear, B.; Watt, D. S.; Gedaly, R. N-Aryl benzenesulfonamide inhibitors of [3H]-thymidine incorporation and beta-catenin signaling in human hepatocyte-derived Huh-7 carcinoma cells. Bioorg Med Chem Lett 2015, 25, 3897-3899.
47. Zhang W, Sviripa V, Kril L M, Chen X, Yu T, Shi J, et al. Fluorinated N,N-dialkylaminostilbenes for Wnt pathway inhibition and colon cancer repression. J Med Chem. 2011; 54(5):1288-97. doi: 10.1021/jm101248v. PubMed PMID: 21291235; PubMed Central PMCID: PMCPMC3073490.
48. Hubbard, W. B.; Joseph, B.; Spry, M.; Vekaria, H. J.; Saatman, K. E.; Sullivan, P. G. Acute Mitochondrial Impairment Underlies Prolonged Cellular Dysfunction after Repeated Mild Traumatic Brain Injuries. J Neurotrauma 2019, 36, 1252-1263.
49. Hubbard, W. B.; Harwood, C. L.; Geisler, J. G.; Vekaria, H. J.; Sullivan, P. G. Mitochondrial uncoupling prodrug improves tissue sparing, cognitive outcome, and mitochondrial bioenergetics after traumatic brain injury in male mice. J Neurosci Res 2018, 96, 1677-1688.
50. Mittal S, El-Serag H B. Epidemiology of hepatocellular carcinoma: consider the population. Journal of clinical gastroenterology. 2013; 47 Suppl:52-6. Epub 2013 May, 2. doi: 10.1097/MCG.0b013e3182872f29. PubMed PMID: 23632345; PubMed Central PMCID: PMCPMC3683119.
51. Society AC. Facts & Figures 2018 Atlanta, Ga. 2018 [cited 2018 Sep. 9]. Available from: https://www.cancer.org/cancer/liver-cancer/about/what-is-key-statistics.htm/#references.
52. Altekruse S F, McGlynn K A, Reichman M E. Hepatocellular carcinoma incidence, mortality, and survival trends in the United States from 1975 to 2005. Journal of clinical oncology: official journal of the American Society of Clinical Oncology. 2009; 27(9):1485-91. Epub 2009/02/20. doi: 10.1200/jco.2008.20.7753. PubMed PMID: 19224838; PubMed Central PMCID: PMCPMC2668555.
53. Dimitroulis D, Damaskos C, Valsami S, Davakis S, Garmpis N, Spartalis E, et al. From diagnosis to treatment of hepatocellular carcinoma: An epidemic problem for both developed and developing world. World journal of gastroenterology: WJG. 2017; 23(29):5282-94. Epub 2017/08/26. doi: 10.3748/wjg.v23.i29.5282. PubMed PMID: 28839428; PubMed Central PMCID: PMCPMC5550777.
54. Lee H C, Kim M, Wands J R. Wnt/Frizzled signaling in hepatocellular carcinoma. Frontiers in bioscience: a journal and virtual library. 2006; 11:1901-15. Epub 2005/12/22. PubMed PMID: 16368566.
55. Wands J R, Kim M. WNT/beta-catenin signaling and hepatocellular carcinoma. Hepatology. 2014; 60(2):452-4. Epub 2014/03/20. doi: 10.1002/hep.27081. PubMed PMID: 24644061.
56. Inagawa S, Itabashi M, Adachi S, Kawamoto T, Hori M, Shimazaki J, et al. Expression and prognostic roles of beta-catenin in hepatocellular carcinoma: correlation with tumor progression and postoperative survival. Clinical cancer research: an official journal of the American Association for Cancer Research. 2002; 8(2):450-6. Epub 2002/02/13. PubMed PMID: 11839663.
57. Sun T, Liu H, Ming L. Multiple Roles of Autophagy in the Sorafenib Resistance of Hepatocellular Carcinoma. Cell Physiol Biochem. 2017; 44(2):716-27. Epub 2017/11/24. doi: 10.1159/000485285. PubMed PMID: 29169150.
58. Cai Z, Qian Z Y, Jiang H, Ma N, Li Z, Liu L Y, et al. hPCL3s Promotes Hepatocellular Carcinoma Metastasis by Activating beta-Catenin Signaling. Cancer research. 2018; 78(10):2536-49. Epub 2018/02/28. doi: 10.1158/0008-5472.can-17-0028. PubMed PMID: 29483096.
59. Shang S, Hua F, Hu Z-W. The regulation of β-catenin activity and function in cancer: therapeutic opportunities. Oncotarget. 2017; 8(20):33972-89. doi: 10.18632/oncotarget.15687. PubMed PMID: PMC5464927.
60. Khramtsov A I, Khramtsova G F, Tretiakova M, Huo D, Olopade O I, Goss K H. Wnt/beta-catenin pathway activation is enriched in basal-like breast cancers and predicts poor outcome. Am J Pathol. 2010; 176(6):2911-20. Epub 2010/04/17. doi: 10.2353/ajpath.2010.091125. PubMed PMID: 20395444; PubMed Central PMCID: PMCPMC2877852.
61. Kobayashi M, Honma T, Matsuda Y, Suzuki Y, Narisawa R, Ajioka Y, et al. Nuclear translocation of beta-catenin in colorectal cancer. Br J Cancer. 2000; 82(10):1689-93. Epub 2000/05/19. doi: 10.1054/bjoc.1999.1112. PubMed PMID: 10817505; PubMed Central PMCID: PMCPMC2374509.
62. Damsky W E, Curley D P, Santhanakrishnan M, Rosenbaum L E, Platt J T, Gould Rothberg B E, et al. beta-catenin signaling controls metastasis in Braf-activated Pten-deficient melanomas. Cancer cell. 2011; 20(6):741-54. Epub 2011/12/17. doi: 10.1016/j.ccr.2011.10.030. PubMed PMID: 22172720; PubMed Central PMCID: PMCPMC3241928.
63. Gekas C, D'Altri T, Aligue R, Gonzalez J, Espinosa L, Bigas A. beta-Catenin is required for T-cell leukemia initiation and MYC transcription downstream of Notch1. Leukemia. 2016; 30(10):2002-10. Epub 2016/04/30. doi: 10.1038/leu.2016.106. PubMed PMID: 27125305.
64. Glick D, Barth S, Macleod K F. Autophagy: cellular and molecular mechanisms. The Journal of pathology. 2010; 221(1):3-12. Epub 2010/03/13. doi: 10.1002/path.2697. PubMed PMID: 20225336; PubMed Central PMCID: PMCPMC2990190.
65. Gozuacik D, Kimchi A. Autophagy as a cell death and tumor suppressor mechanism. Oncogene. 2004; 23(16):2891-906. Epub 2004/04/13. doi: 10.1038/sj.onc.1207521. PubMed PMID: 15077152.
66. Poillet-Perez L, Despouy G, Delage-Mourroux R, Boyer-Guittaut M. Interplay between ROS and autophagy in cancer cells, from tumor initiation to cancer therapy. Redox Biology. 2015; 4:184-92. doi: 10.1016/j.redox.2014.12.003. PubMed PMID: 25590798; PubMed Central PMCID: PMCPMC4803791.
67. Shimizu S, Takehara T, Hikita H, Kodama T, Tsunematsu H, Miyagi T, et al. Inhibition of autophagy potentiates the antitumor effect of the multikinase inhibitor sorafenib in hepatocellular carcinoma. Int J Cancer. 2012; 131(3):548-57. doi: 10.1002/ijc.26374. PubMed PMID: 21858812.
68. Shi Y H, Ding Z B, Zhou J, Hui B, Shi G M, Ke A W, et al. Targeting autophagy enhances sorafenib lethality for hepatocellular carcinoma via ER stress-related apoptosis. Autophagy. 2011; 7(10):1159-72. Epub 2011/06/22. doi: 10.4161/auto.7.10.16818. PubMed PMID: 21691147.
69. Chi K H, Wang Y S, Huang Y C, Chiang H C, Chi M S, Chi C H, et al. Simultaneous activation and inhibition of autophagy sensitizes cancer cells to chemotherapy. Oncotarget. 2016; 7(36):58075-88. Epub 2016/08/04. doi: 10.18632/oncotarget.10873. PubMed PMID: 27486756; PubMed Central PMCID: PMCPMC5295413.
70. Liu L, Zhi Q, Shen M, Gong F R, Zhou B P, Lian L, et al. FH535, a β-catenin pathway inhibitor, represses pancreatic cancer xenograft growth and angiogenesis. Oncotarget. 2016; 7(30):47145-62. doi: 10.18632/oncotarget.9975. PubMed PMID: 27323403; PubMed Central PMCID: PMCPMC5216931.
71. Shiah S G, Shieh Y S, Chang J Y. The Role of Wnt Signaling in Squamous Cell Carcinoma. Journal of dental research. 2016; 95(2):129-34. Epub 2015/10/31. doi: 10.1177/0022034515613507. PubMed PMID: 26516128.
72. Turcios L, Vilchez V, Acosta L F, Poyil P, Butterfield D A, Mitov M, et al. Sorafenib and FH535 in combination act synergistically on hepatocellular carcinoma by targeting cell bioenergetics and mitochondrial function. Dig Liver Dis. 2017; 49(6):697-704. doi: 10.1016/j.dld.2017.01.146. PubMed PMID: 28179093.
73. Galuppo R, Maynard E, Shah M, Daily M F, Chen C, Spear B T, et al. Synergistic inhibition of HCC and liver cancer stem cell proliferation by targeting RAS/RAF/MAPK and WNT/beta-catenin pathways. Anticancer research. 2014; 34(4):1709-13. Epub 2014/04/03. PubMed PMID: 24692700; PubMed Central PMCID: PMCPMC5733784.
74. Nakabayashi H, Taketa K, Miyano K, Yamane T, Sato J. Growth of human hepatoma cells lines with differentiated functions in chemically defined medium. Cancer research. 1982; 42(9):3858-63. PubMed PMID: 6286115.
75. Gedaly R, Galuppo R, Daily M F, Shah M, Maynard E, Chen C, et al. Targeting the Wnt/beta-catenin signaling pathway in liver cancer stem cells and hepatocellular carcinoma cell lines with FH535. PloS one. 2014; 9(6):e99272. Epub 2014/06/19. doi: 10.1371/journal.pone.0099272. PubMed PMID: 24940873; PubMed Central PMCID: PMCPMC4062395.
76. Ullman-Cullere M H, Foltz C J. Body condition scoring: a rapid and accurate method for assessing health status in mice. Laboratory animal science. 1999; 49(3):319-23. Epub 1999/07/14. PubMed PMID: 10403450.
77. Turcios L, Vilchez V, Acosta L F, Poyil P, Butterfield D A, Mitov M, et al. Sorafenib and FH535 in combination act synergistically on hepatocellular carcinoma by targeting cell bioenergetics and mitochondrial function. Digestive and Liver Disease. doi: 10.1016/j.d1d.2017.01.146.
78. Kril L M, Vilchez V, Jiang J, Turcios L, Chen C, Sviripa V M, et al. N-Aryl benzenesulfonamide inhibitors of [3H]-thymidine incorporation and beta-catenin signaling in human hepatocyte-derived Huh-7 carcinoma cells. Bioorganic & medicinal chemistry letters. 2015; 25(18):3897-9. Epub 2015/08/06. doi: 10.1016/j.bmc1.2015.07.040. PubMed PMID: 26243371; PubMed Central PMCID: PMCPMC4540627.
79. Gedaly R, Angulo P, Hundley J, Daily M F, Chen C, Koch A, et al. PI-103 and sorafenib inhibit hepatocellular carcinoma cell proliferation by blocking Ras/Raf/MAPK and PI3K/AKT/mTOR pathways. Anticancer research. 2010; 30(12):4951-8. PubMed PMID: 21187475; PubMed Central PMCID: PMC3141822.
80. Petherick K J, Williams A C, Lane J D, Ordonez-Moran P, Huelsken J, Collard T J, et al. Autolysosomal beta-catenin degradation regulates Wnt-autophagy-p62 crosstalk. The EMBO journal. 2013; 32(13):1903-16. doi: 10.1038/emboj.2013.123. PubMed PMID: 23736261; PubMed Central PMCID: PMCPMC3981178.
81. Lida J, Dorchak J, Lehman J R, Clancy R, Luo C, Chen Y, et al. FH535 inhibited migration and growth of breast cancer cells. PloS one. 2012; 7(9):e44418. Epub 2012/09/18. doi: 10.1371/journal.pone.0044418. PubMed PMID: 22984505; PubMed Central PMCID: PMCPMC3439405.
82. Chen Y, Rao X, Huang K, Jiang X, Wang H, Teng L. FH535 Inhibits Proliferation and Motility of Colon Cancer Cells by Targeting Wnt/β-catenin Signaling Pathway. Journal of Cancer. 2017; 8(16):3142-53. doi: 10.7150/jca.19273. PubMed PMID: 29158786; PubMed Central PMCID: PMCPMC5665030.
83. Su H, Jin X, Zhang X, Zhao L, Lin B, Li L, et al. FH535 increases the radiosensitivity and reverses epithelial-to-mesenchymal transition of radioresistant esophageal cancer cell line KYSE-150R. Journal of translational medicine. 2015; 13:104. Epub 2015/04/19. doi: 10.1186/s12967-015-0464-6. PubMed PMID: 25888911; PubMed Central PMCID: PMCPMC4384308.
84. Lee J, Giordano S, Zhang J. Autophagy, mitochondria and oxidative stress: cross-talk and redox signalling. The Biochemical journal. 2012; 441(Pt 2):523-40. doi: 10.1042/bj20111451. PubMed PMID: 22187934; PubMed Central PMCID: PMCPMC3258656.
85. Gao C, Xiao G, Hu J. Regulation of Wnt/β-catenin signaling by posttranslational modifications. Cell Biosci. 2014; 4:13. doi: 10.1186/2045-3701-4-13. PubMed PMID: 24594309; PubMed Central PMCID: PMCPMC3977945.
86. Jia Z, Wang J, Wang W, Tian Y, XiangWei W, Chen P, et al. Autophagy eliminates cytoplasmic beta-catenin and NICD to promote the cardiac differentiation of P19CL6 cells. Cellular signalling. 2014; 26(11):2299-305. Epub 2014/08/08. doi: 10.1016/j.cellsig.2014.07.028. PubMed PMID: 25101857.
87. Su N, Wang P, Li Y. Role of Wnt/beta-catenin pathway in inducing autophagy and apoptosis in multiple myeloma cells. Oncology letters. 2016; 12(6):4623-9. doi: 10.3892/01.2016.5289. PubMed PMID: 28105169; PubMed Central PMCID: PMCPMC5228543.
88. Kuhn K, Cott C, Bohler S, Aigal S, Zheng S, Villringer S, et al. The interplay of autophagy and beta-Catenin signaling regulates differentiation in acute myeloid leukemia. Cell Death Discov. 2015; 1:15031. doi: 10.1038/cddiscovery.2015.31. PubMed PMID: 27551462; PubMed Central PMCID: PMCPMC4979480.
89. Fu Y, Chang H, Peng X, Bai Q, Yi L, Zhou Y, et al. Resveratrol inhibits breast cancer stem-like cells and induces autophagy via suppressing Wnt/beta-catenin signaling pathway. PloS one. 2014; 9(7):e102535. Epub 2014/07/30. doi: 10.1371/journal.pone.0102535. PubMed PMID: 25068516; PubMed Central PMCID: PMCPMC4113212.
90. Tao H, Chen F, Liu H, Hu Y, Wang Y, Li H. Wnt/β-catenin signaling pathway activation reverses gemcitabine resistance by attenuating Beclin1-mediated autophagy in the MG63 human osteosarcoma cell line. Molecular medicine reports. 2017; 16(2):1701-6. doi: 10.3892/mmr.2017.6828. PubMed PMID: 28656199; PubMed Central PMCID: PMCPMC5562091.
91. Lin R, Feng J, Dong S, Pan R, Zhuang H, Ding Z. Regulation of autophagy of prostate cancer cells by beta-catenin signaling. Cell Physiol Biochem. 2015; 35(3):926-32. doi: 10.1159/000369749. PubMed PMID: 25633614.
92. Gao C, Cao W, Bao L, Zuo W, Xie G, Cai T, et al. Autophagy negatively regulates Wnt signalling by promoting Dishevelled degradation. Nat Cell Biol. 2010; 12(8):781-90. doi: 10.1038/ncb2082. PubMed PMID: 20639871.
93. Vanmeerbeek et al. Trial watch: chemotherapy-induced immunogenic cell death in immuno-oncology. Oncoimmunology 9: e1703449, 2020.
94. Legrand et al. The Diversification of Cell Death and Immunity: Memento Mori. Mol Cell 76:

232-242, 2019.
95. Zhou et al. Immunogenic cell death in cancer therapy: Present and emerging inducers. J Cell Mol Med. 23: 4854-4865, 2019.
96. Zhang W, Sviripa V, Kril K, Yu T, Xie Y, Hubbard W, Sullivan P, Chen X, Zhan C, Yang-Hartwich Y, Evers B M, Spear B, Gedaly R, Watt D and Liu C. An Underlying Mechanism of Dual Wnt Inhibition and AMPK Activation: Mitochondrial Uncouplers Masquerading as Wnt Inhibitors, J Med Chem. 62: 11348-11358, 2019.
97. Kenji Chamoto, Partha S. Chowdhury, Alok Kumar, Kazuhiro Sonomura, Fumihiko Matsuda, Sidonia Fagarasan, and Tasuku Honjo. Mitochondrial activation chemicals synergize with surface receptor PD-1 blockade for T cell-dependent antitumor activity. PNAS 114: E761-E770, 2017.

1. A method of treating a disease, the method comprising administering one or more N-aryl benezenesulfonamides or analogs thereof to a subject in need thereof.

2. The method of claim 1, wherein the subject is a human subject.

3. The method of claim 1, wherein the N-aryl benzenesulfonamide is a proton uncoupler.

4. The method of claim 1, wherein the N-aryl benzenesulfonamide is a halogenated N-aryl benzenesulfonamide.

5. The method of claim 1, wherein the N-aryl benzenesulfonamide is selected from the group consisting of 2,5-dichloro-N-(2-methyl-4-nitrophenyl)benzenesulfonamide (FH535), 2,5-dichloro-N-(4-nitronaphthalen-1-yl)benzenesulfonamide (Y3), analogs thereof, and combinations thereof.

6. The method of claim 4, wherein the N-aryl benzenesulfonamide is Y3.

7. The method of claim 1, wherein the disease is cancer.

8. The method of claim 7, wherein the cancer is characterized by aberrant Wnt/β-catenin signaling.

9. The method of claim 8, wherein the cancer is selected from the group consisting of hepatocellular cancer, colorectal cancer, or a combination thereof.

10. The method of claim 7, wherein the cancer is ovarian cancer.

11. The method of claim 1, wherein the disease is traumatic brain injury.

12. The method of claim 1, wherein the disease is a bacterial disease.

13. The method of claim 1, wherein the disease is a metabolic disease.

14. The method of claim 1, wherein the one or more N-aryl benezenesulfonamides or analogs thereof are administered as part of a composition.

15. The method of claim 14, wherein the composition comprises the one or more N-aryl benezenesulfonamides or analogs thereof and a pharmaceutically acceptable solvent or carrier.


SEQUENCE LISTING (12 sequences)

SEQ ID NO: 1 - DNA, 19 bp, Artificial
  p62 RT-PCR primer
aagccgggtgggaatgttg

SEQ ID NO: 2 - DNA, 19 bp, Artificial
  p62 RT-PCR primer
gcttggcccttcggattct

SEQ ID NO: 3 - DNA, 23 bp, Artificial
  c-MYC RT-PCR primer
ttttcgggtagtggaaaaccagc

SEQ ID NO: 4 - DNA, 22 bp, Artificial
  c-MYC RT-PCR primer
agtagaaatacggctgcaccga

SEQ ID NO: 5 - DNA, 19 bp, Artificial
  Survivin RT-PCR primer
caaggagctggaaggctgg

SEQ ID NO: 6 - DNA, 22 bp, Artificial
  Survivin RT-PCR primer
gttcttggctctttctctgtcc

SEQ ID NO: 7 - DNA, 22 bp, Artificial
  AXIN 2 RT-PCR primer
cagcagagggacaggaatcatt

SEQ ID NO: 8 - DNA, 23 bp, Artificial
  AXIN 2 RT-PCR primer
gccagtttctttggctctttgtg

SEQ ID NO: 9 - DNA, 19 bp, Artificial
  Cyclin D1 RT-PCR primer
ggatgctggaggtctgcga

SEQ ID NO: 10 - DNA, 22 bp, Artificial
  Cyclin D1 RT-PCR primer
tagaggccacgaacatgcaagt

SEQ ID NO: 11 - DNA, 23 bp, Artificial
  b2-microglobulin RT-PCR primer
gactttgtcacagcccaagatag

SEQ ID NO: 12 - DNA, 23 bp, Artificial
  b2-microglobulin RT-PCR primer
tccaatccaaatgcggcatcttc